(19) United States
(12) Patent Application Publication
(10) Pub. No.: US 2026/0159522 A1
(43) Pub. Date: Jun. 11, 2026

(54) PROCESS FOR THE PREPARATION OF A CHIRAL PYRROLO TRIAZOLE ALCOHOL

(71) Applicant: Hoffmann-La Roche Inc., Little Falls, NJ (US)

(72) Inventors: Stephan BACHMANN, Allschwil (CH); Raphael BIGLER, Frauenfeld (CH); Serena BISAGNI, Basel (CH); Dainis KALDRE, Loerrach (DE); Christian Oliver KAPPE, Graz (AT); Michael PRIESCHL, Graz (AT); Kurt PUENTENER, Kaiseraugst (CH); Rosa Maria RODRIGUEZ SARMIENTO, Basel (CH); Joerg SEDELMEIER, Basel (CH); Jason Douglas WILLIAMS, Graz (AT)

(73) Assignee: Hoffmann-La Roche Inc., Little Falls, NJ (US)

(21) Appl. No.: 19/415,612

(22) Filed: Dec. 10, 2025

Related U.S. Application Data
(63) Continuation of application No. PCT/EP2024/066839, filed on Jun. 17, 2024.

(30) Foreign Application Priority Data
Jun. 19, 2023 (EP) 23180069.9

(51) Int. Cl. C07D 487/04; A61K 31/4196; B01J 31/12

(52) U.S. Cl. CPC C07D 487/04; A61K 31/4196; B01J 31/12; B01J 2531/821; B01J 2531/827

(57) ABSTRACT

The invention relates to a novel process for the preparation of chiral pyrrolo triazole alcohols of the formula I

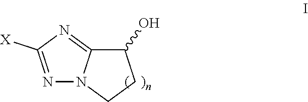

wherein X is a halogen atom, n is an integer of 1, 2 or 3 and wherein the spiral bond “ [symbol] ” stands for “ [symbol] ” or for “ [symbol] ” or mixtures of the enantiomers.

The chiral pyrrolo triazole alcohols of the formula I are versatile intermediates for the preparation of compounds that have the potential to serve as active pharmaceutical ingredients in drugs.

The invention relates to a novel process for the preparation of a chiral pyrrolo triazole alcohol of the formula I

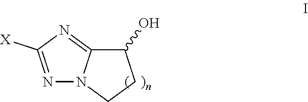

wherein X is a halogen atom and n is an integer of 1, 2 or 3 and the spiral bond “ [symbol] ” stands for “ [symbol] ” or for “ [symbol] ” or mixtures of the enantiomers.

The chiral pyrrolo triazole alcohols of the formula I are versatile intermediates for the preparation of compounds that have the potential to serve as active pharmaceutical ingredients in drugs. For instance chiral pyrrolo triazole alcohols of the formula I can be used as intermediates for the preparation of compounds that have the potential to act as gamma-secretase modulators as disclosed in the International Patent Publication WO 2020/120521), for the preparation of compounds that have the potential to act as melanocortin 4 receptor antagonists as disclosed in the International Patent Publication WO2021250541 or for the preparation of compounds that have the potential to act as ubiquitin-specific-processing proteases 1 as disclosed in the International Patent Publication WO 2021/247606.

The invention further relates to novel chiral pyrrolo triazole alcohols of the formula I

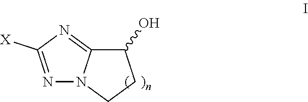

wherein X is a halogen atom and n is an integer of 1, 2 or 3 and the spiral bond “ [symbol] ” stands for “ [symbol] ” or for “ [symbol] ” or mixtures of the enantiomers.

The object of the present invention was to find a suitable process for the preparation of this versatile chiral pyrrolo triazole alcohol compounds of formula I.

The object could be reached with the process as outlined below.

The process for the preparation of a chiral pyrrolo triazole alcohol of the formula I

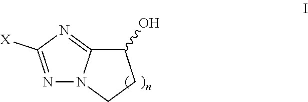

wherein X is a halogen atom, n is an integer of 1, 2 or 3 and wherein the spiral bond “ [symbol] ” stands for “ [symbol] ” or for “ [symbol] ” or mixtures of the enantiomers comprises the reduction of a ketone of the formula V

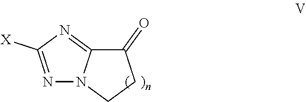

wherein X and n are as above with
a) a metal complex catalyst in the presence a reducing agent or
b) an oxidoreductase enzyme
to form the chiral alcohol of the formula I.

The following definitions are set forth to illustrate and define the meaning and scope of the various terms used to describe the invention herein.

The term “C₁₋₆-alkyl” relates to a branched or straight-chain monovalent saturated aliphatic hydrocarbon radical of one to six carbon atoms, preferably one to four, more preferably one to two carbon atoms. This term is further exemplified by radicals as methyl, ethyl, n-propyl, i-propyl, n-butyl, i-butyl, s-butyl or t-butyl, pentyl and its isomers or hexyl and its isomers.

The term “C₁₋₆-alkoxy” refers to a C₁₋₆-alkyl group as defined above to which an oxygen atom is attached.

The term “halogen” refers to fluorine, chlorine, bromine or iodine, but particularly to chlorine and bromine.

The term “aryl”, relates to an aromatic carbon ring such as to the phenyl or naphthyl ring, preferably the phenyl ring.

The term “heteroaryl” refers to an aromatic 5 to 6 membered monocyclic ring or 9 to 10 membered bicyclic ring which can comprise 1, 2 or 3 heteroatoms selected from nitrogen, oxygen and/or sulphur, such as pyridinyl, pyrazolyl, pyrimidinyl, benzoimidazolyl, quinolinyl and isoquinolinyl.

The spiral bond “ [symbol] ” stands for “ [symbol] ” or for “ [symbol] ” and thus indicating chirality of the molecule, but also for mixtures of the enantiomers.

Whenever a chiral carbon is present in a chemical structure, it is intended that all stereoisomers associated with that chiral carbon are encompassed by the structure as pure stereoisomers as well as mixtures thereof.

The process of the present invention can be illustrated with Scheme 1 below

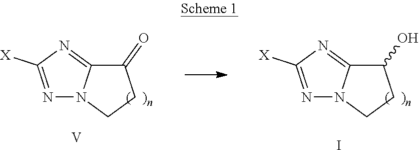

wherein X is halogen and n is 1, 2 or 3 and the spiral bond “ [symbol] ” stands for “ [symbol] ” or for “ [symbol] ” or mixtures of the enantiomers.

The reduction can be accomplished with
a) a metal complex catalyst in the presence of a reducing agent or
b) an oxidoreductase enzyme.

The ketone of formula V can be prepared in accordance with Schemes 2 and 3 below

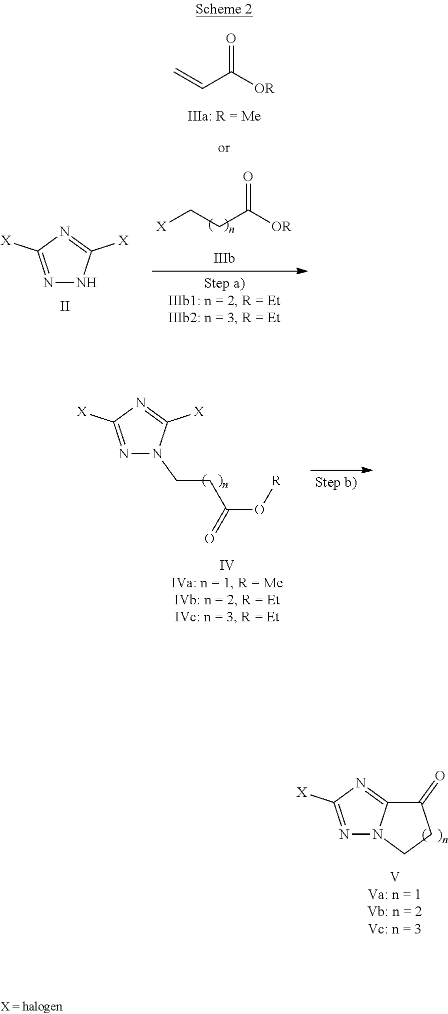

The process comprises in a first step
a) transforming a 3,5-dihalogen-1H-1,2,4-triazole of formula II

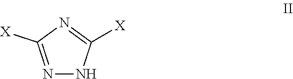

wherein X is a halogen atom, either by
a¹) a Michael addition using an acryl ester IIIa.

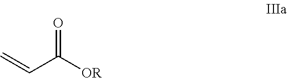

wherein R is C₁₋₄alkyl or by
a²) an alkylation with a halogen carboxylic acid alkyl ester of formula IIIb

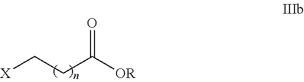

wherein R is C₁₋₄ alkyl, n is an integer of 1, 2 or 3 and X is halogen,
into a 3,5-dihalogen 1,2,4-triazole-carboxylic acid ester of formula IV

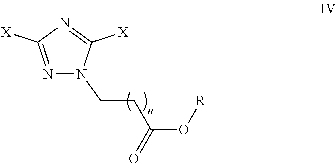

wherein R, n and X are as above and in a second step
b) a ring closure of the 3,5-dihalogen 1,2,4-triazole-carboxylic acid ester of formula IV with organometallic reagents.

The 3,5-dihalogen-1H-1,2,4-triazole stating compounds, preferably the 3,5-dibromo-1H-1,2,4-triazole are commercially available.

The Michael addition using an acryl ester IIIa and the alkylation with a halogen carboxylic acid alkyl ester of formula IIIb is typically performed in the presence of base which can be selected from organic bases such as triethylamine, N,N-diisopropylethylamine, tributylamine and inorganic bases such as potassium carbonate or cesium carbonate.

Triethylamine is a preferred base for the Michael addition using an acryl ester IIIa and potassium carbonate is the preferred base for alkylation with a halogen carboxylic acid alkyl ester of formula IIIb.

Suitable acryl ester IIIa is the C₁₋₄ alkyl ester, preferably the methyl ester and suitable halogen carboxylic acid alkyl ester of formula IIIb is the bromo-C₁₋₄ alkyl ester, preferably the methyl ester or the ethyl ester.

The reaction can be performed in a suitable solvent selected from an alcoholic solvent (e.g. methanol, ethanol, iso-propanol) or a polar aprotic solvent (e.g. DMSO, acetonitrile, THF, MeTHF) at a reaction temperature between 30° C. and 170° C., preferably in MeTHF at 70° C.

N-Alkylation of the N-containing heterocycles is reported in the literature (Gmach J. et. al., Synthesis, 2016, 48, 2681-2704).

The 3,5-dihalogen 1,2,4-triazole-carboxylic acid ester of formula IV

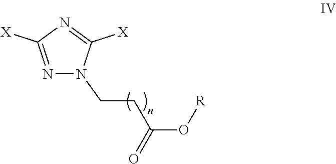

wherein R is C₁₋₄ alkyl, n is an integer of 1, 2 or 3 and X is halogen are novel compounds and therefore constitute a further embodiment of the invention.

In preferred embodiment X is bromine.

In a further preferred embodiment R is methyl or ethyl.

Particular preferred 3,5-dihalogen 1,2,4-triazole-carboxylic acid esters of formula IV are those with
X=bromine, R=methyl and n is 1;
X=bromine, R=ethyl and n is 1;
X=bromine, R=methyl and n is 2;
X=bromine, R=ethyl and n is 2.

In step b) the 3,5-dihalogen 1,2,4-triazole-carboxylic acid ester of formula IV can be subjected to a ring closure reaction with organometallic reagents such as organo lithium or organo magnesium compounds.

Suitable organo lithium compounds are n-hexyllithium, n-butyllithium, phenyllithium or methyllithium.

Suitable organo magnesium compounds are methylmagnesium bromide, ethylmagnesium chloride, ethylmagnesium bromide, isopropylmagnesium bromide or isopropylmagnesium chloride.

The reaction can be performed in a suitable solvent selected from 2-methyl tetrahydrofuran, tetrahydrofuran, toluene or methyl t-butyl ether at a reaction temperature between 0° C. and 100° C.

The reaction requires a quenching with an acid. Suitable quench acid is acetic acid or citric acid.

Suitable reaction technology is a batch reactor set-up, a plug flow reactor set-up or, as a preferred technology, a continuous-stirred-tank reactor (CSTR) set-up.

Ring expansion can be accomplished following Scheme 3 below:

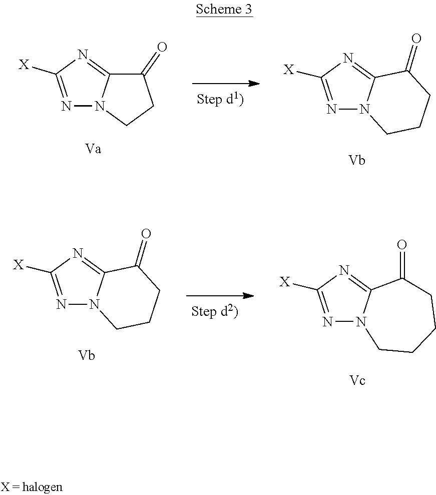

The homologation or ring enlargement of cyclic ketones can happen by using diazo compounds (e.g. diazomethane or trimethylsilyldiazomethane,) in the presence of a promoter such as a Lewis acid (e.g. BF₃·Et₂O, AlMe₃).

In step d¹) this results in the ring expansion of the bicyclic 5 membered ring ketone Va to the bicyclic cyclohexyl ketone Vb.

In step d²) the bicyclic 6 membered ring ketone Vb, can be homologated to the 7 membered ring bicyclic ketone under similar experimental conditions.

Homologation of cyclic or non-cyclic ketones under different conditions is reported in literature (Candeias et al, Chem, Rev, 2016, 2937-2981).

a) Reduction of Ketone of Formula V with Metal Complex Catalyst:

Suitable metal complex catalysts for the reduction of the ketone of formula V are a ruthenium or iridium complex catalysts.

They can be selected from a variety of ruthenium or iridium catalyst complexes as outlined below and include isomers and mixtures thereof.

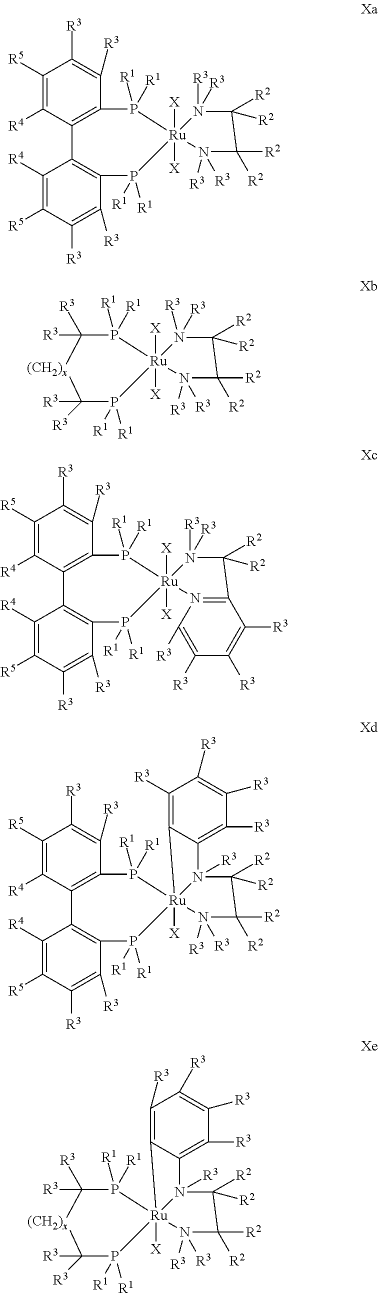

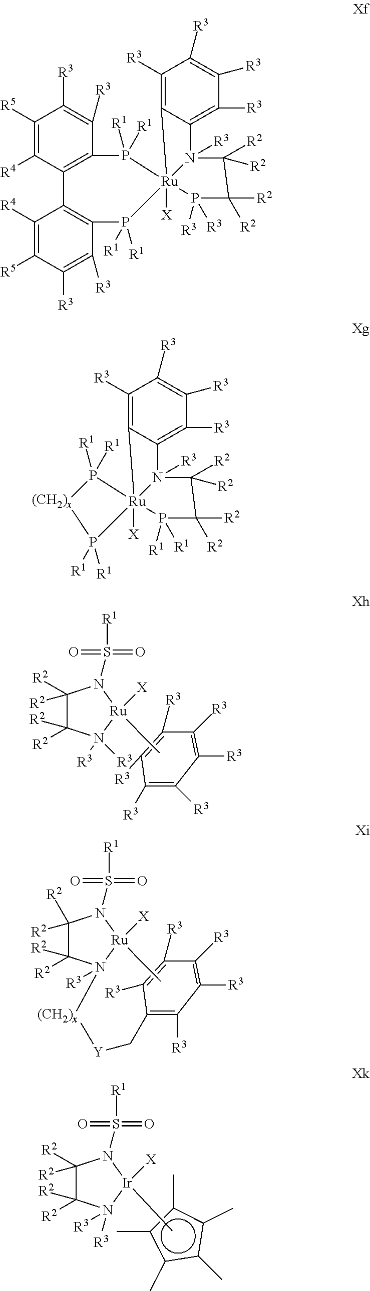

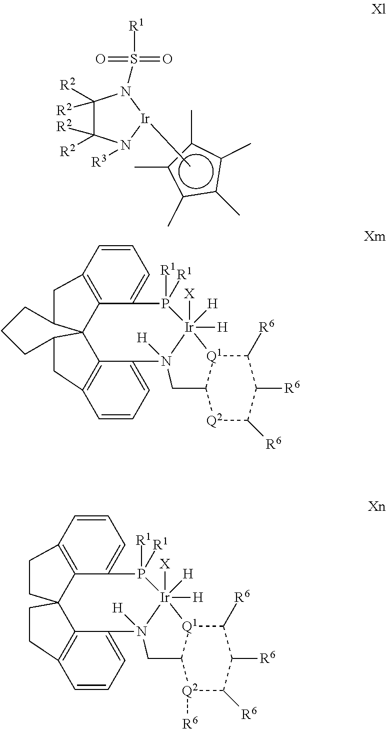

wherein, for each individual structure and independent of each other,
R¹ is, independently of each other, C₁₋₆-alkyl, C₄₋₆-cycloalkyl, phenyl or heteroaryl, optionally substituted with one or more C₁₋₆-alkyl or C₁₋₆-alkoxy;
R² is, independently of each other, hydrogen, C₁₋₆-alkyl, C₄₋₆-cycloalkyl, phenyl or heteroaryl, optionally substituted with one or more C₁₋₆-alkyl or C₁₋₆-alkoxy or two R² taken together form a ring bridged with a —(CH₂)₄— unit;
R³ is, independently of each other, hydrogen or C₁₋₆-alkyl, C₄₋₆-cycloalkyl, phenyl or heteroaryl, optionally substituted with one or more C₁₋₆-alkyl or C₁₋₆-alkoxy;
R⁴ is, independently of each other, hydrogen, C₁₋₆-alkyl, C₄₋₆-cycloalkyl, phenyl or heteroaryl, optionally substituted with one or more C₁₋₆-alkyl or C₁₋₆-alkoxy or both R⁴ are taken together to form a ring bridged with a —O—(CH₂)ₓ—O— unit;
R⁵ is, independently of each other, hydrogen, C₁₋₆-alkyl, C₄₋₆-cycloalkyl, phenyl or heteroaryl, optionally substituted with one or more C₁₋₆-alkyl or C₁₋₆-alkoxy or both neighboring R⁴ and R⁵ are taken together to form a ring bridged with a —(CH)₄— unit or a —O—(CH₂)x-O— unit;
R⁶ is, independently of each other, hydrogen or C₁₋₆-alkyl;
X is either a coordinated ligand or a counter anion selected from halogen, C₁₋₆-alkoxy, tetrahalogenoborate, tetrakis (3,5-bis(trihalogeno-C₁₋₆-alkyl) phenyl)borate, acetylacetonate, hexahalogenophosphate, p-tolylsulfonate, methansulfonate or trihalogeno methanesulfonate; Y is oxygen or —CH₂—;
x is 1, 2 or 3;
and the dotted ring signifies an aromatic ring when Q¹ is nitrogen and Q² is carbon;
and the dotted ring signifies a cycloalkane ring wherein Q¹ and Q² are sulfur.

In a preferred embodiment the formulas Xh, Xk, Xm or Xn are selected.

Preferred substituents for Xh, and Xk are:
R¹ is, independently of each other, C₁₋₆-alkyl, phenyl, optionally substituted with one or more C₁₋₆-alkyl or C₁₋₆-alkoxy;
R² is, independently of each other, hydrogen, phenyl, optionally substituted with one or more C₁₋₆-alkyl or two R² taken together form a ring bridged with a —(CH₂)₄— unit;
R³ is, independently of each other, hydrogen or C₁₋₆-alkyl, phenyl, optionally substituted with one or more C₁₋₆-alkyl;
X is either a coordinated ligand or a counter anion selected from halogen, tetrafluoroborate, tetrakis (3,5-bis(trifluoromethyl)phenyl)borate, hexafluorophosphate or trifluoromethanesulfonate.

Preferred substituents for Xm and Xn are:
R¹ is, independently of each other, phenyl, optionally substituted with one or more C₁₋₆-alkyl or C₁₋₆-alkoxy;
R⁶ is, independently of each other, hydrogen or C₁₋₆-alkyl;
X is a coordinated ligand selected from halogen
and the dotted ring signifies an aromatic ring when Q¹ is nitrogen and Q² is carbon.

In a further preferred embodiment, substituents for Xh, and Xk are:
R¹ is independently of each other, methyl, phenyl, optionally substituted with one or more C₁₋₆-alkyl;
R² is, independently of each other, hydrogen, phenyl or two R² taken together form a ring bridged with a —(CH₂)₄— unit;
R³ is, independently of each other, hydrogen or C₁₋₆-alkyl;
X is either a coordinated ligand or a counter anion selected from chloride or trifluoromethanesulfonate.

Further preferred embodiment substituents for Xm and Xn are:
R¹ is phenyl, optionally substituted with one or more C₁₋₆-alkyl;
R⁶ is, independently of each other, hydrogen, tert.-butyl or methyl;
X is chloride
and the dotted ring signifies an aromatic ring when Q¹ is nitrogen and Q² is carbon.

Suitable catalysts are typically commercially available e.g. from Jiuzhou Pharma, Sinocompound or Johnson Matthey or catalogue suppliers such as e.g. Strem or Sigma Aldrich.

The reduction of the ketone of formula V can be performed in the presence of reducing agent and a suitable organic solvent.

One option for the reducing agent is using a mixture of formic acid and a trialkylamine, preferably triethylamine or an alkali formate, like sodium formate, and the presence of a tetraalkyl ammoniumhalogenide, such as tetrabutyl ammoniumbromide (TBAB).

Mixing ratios can vary e.g. between 1 to 5 eq. of formic acid and 1 to 5 eq. of triethylamine or e.g. 5 eq. of sodium formate and 0.5 eq. of TBAB.

Typically an organic solvent selected from e.g. ethanol, toluene, acetonitrile, 2-methyltetrahydrofuran or propylene carbonate can be used.

The reaction temperature is selected between 10° C. and 100° C., preferably between 20° C. and 50° C.

In a further option the reduction takes place in the presence of hydrogen at a hydrogen pressure of 1 bar to 100 bar, preferably of 60 bar to 80 bar and at a reaction temperature of 10° C. to 100° C., preferably of 40° C. to 60° C.

Suitable organic solvents are aliphatic alcohols such as ethanol.

There are catalysts which require a base for activation.

Suitable bases are inorganic bases selected from alkali or earth alkali-carbonates or -hydrogen carbonates or phosphates or hydrogenphosphates or dihydrogenphosphates or acetates or formates or organic bases selected from amines, alkali alcoholates or amidines. Organic bases are usually preferred. Typical representatives of organic bases are potassium tert-butylate or 1,8-diazabicyclo[5.4.0]undec-7-ene (DBU), 1,4-diazabicyclo(2.2.2)octane (DABCO) and 7-methyl-1,5,7-triazabicyclo[4.4.0]dec-5-ene (MTBD), most preferred is potassium tert-butylate.

The chiral pyrrolo triazole alcohol of formula I can be separated from the reaction mixture by evaporation of the solvent. Subsequent column chromatography and/or product crystallization renders the chiral alcohol of formula I in good yields, high purity and high enantiomeric excess.

b) Reduction of Ketone of Formula V with an Oxidoreductase:

Suitable oxidoreductases are selected from those which are capable to reduce the ketone of formula V

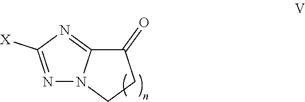

wherein X is a halogen atom, n is an integer of 1, 2 or 3 and to form the chiral pyrrolo triazole alcohol of the formula I

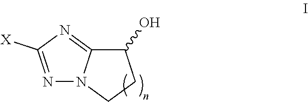

wherein X and n are as above and wherein the spiral bond “ [symbol] ” stands for “ [symbol] ” or for “ [symbol] ” or mixtures of the enantiomers.
with an enantiomeric excess of at least 90%, preferably at least 95%, more preferably at least 98%.

The asymmetric reduction is catalyzed by an oxidoreductase, usually in the presence of NADH or NADPH as cofactor, which is regenerated in-situ. NAD⁺ or NADP⁺ are added to the reaction, rather than their reduced counterparts. The substrate to cofactor ratio (S/C) of NAD⁺ or NADP⁺ is usually kept in a range between 5 and 1000, preferably between 10 and 500.

The oxidized cofactor is as a rule continuously regenerated with a secondary alcohol as cosubstrate. Typical cosubstrates can be selected from 2-propanol, 2-butanol, pentan-1,4-diol, 2-pentanol, 4-methyl-2-pentanol, 2-heptanol, hexan-1,5-diol, 2-heptanol or 2-octanol, preferably 2-propanol. Preferably, the cofactor is regenerated by means of the cosubstrate at the same enzyme also catalyzing the target reaction. The acetone formed when 2-propanol is used as cosubstrate is in a further preferred embodiment continuously removed from the reaction mixture.

Also well-known is the cofactor regeneration via an additional enzyme oxidizing its natural substrate and providing the reduced cofactor. For example secondary alcohol dehydrogenase/alcohol; glucose dehydrogenase/glucose; formate dehydrogenase/formic acid; glucose-6-phosphate dehydrogenase/glucose-6-phosphate; phosphite dehydrogenase/phosphite; hydrogenase/molecular hydrogen and the like. In addition electrochemical regeneration methods are known as well as chemical cofactor regeneration methods comprising a metal catalyst and a reducing agent are suitable. In particular, when glucose dehydrogenase/glucose is used for cofactor regeneration, the pH has to be maintained by controlled addition of a base to neutralize the formed gluconic acid—the oxidized by-product of the reduced nicotinamide cofactor regeneration. The substrate to coenzyme ratio (S/GDH) is usually kept in a range between 5 and 1000, preferably between 10 and 200.

Preferred microbial oxidoreductase enzymes origin from yeasts, bacteria or from mammalian cells.

The oxidoreductase can be applied in the form of the isolated enzyme(s) or as whole cells, optionally in immobilized form by one of the numerous conventional methods described in literature.

In a particular embodiment of the present invention, the asymmetric reduction is performed in an aqueous medium in the presence of an organic cosolvent which can be selected for example from glycerol, 2-propanol, dimethyl sulfoxide, diethylether, tert-butylmethylether, diisopropylether, dibutylether, toluene, 2-methyltetrahydrofuran, ethylacetate, butylacetate, heptane, hexane or cyclohexene or from mixtures thereof.

The presence of an organic cosolvent is particularly advantageous as a homogenous suspension can be formed which allows simple separation of the desired ketone of formula V by filtration.

The reaction concentration (concentration of ketone of formula V and chiral alcohol of formula I in the reaction mixture) is usually kept in a range between 1% and 25% w/v, preferably between 4% and 20% w/v.

A buffer salt is used in the reaction, which can be selected for example from potassium phosphate buffer, Tris HCl buffer, bicine buffer, HEPES buffer, PIPES buffer. The pH of the reaction is kept in a range between 6.0 and 9.0, preferably between 6.5 and 7.5.

The reaction temperature is usually kept in a range between 1° and 50° C., preferably between 2° and 30° C.

The substrate to enzyme ratio (S/E) is usually kept in a range between 5 and 1000, preferably between 10 and 200.

Upon termination of the reaction (as a rule >90% conversion) the product is conventionally worked up by extraction.

Depending on the ketone substrate the preferred catalyst/cofactor/cosubstrate systems can vary.

For the formation of the (S)-2-bromo-6,7-dihydro-5H-pyrrolo[1,2-b][1,2,4]triazole-7-ol of formula (S)-Ia the following oxidoreductases have been proved to be useful.

NADPH-dependent oxidoreductases can be selected from types KRED-NADPH-130, KRED-P1-C01, KRED-P2-D11, KRED-P2-D12, KRED-463 from Codexis Inc, or NADH dependent oxidoreductases can be selected from types ADH-109. ADH-132 and ADH-172 from c-LEcta.

For the formation of the (R)-2-bromo-6,7-dihydro-5H-pyrrolo[1,2-b][1,2,4]triazole-7-ol of the formula (R)-Ia the following oxidoreductases have been proved to be useful.

NADPH-dependent oxidoreductases can be selected from types ADH-61 from Johnson Matthey or NADH dependent oxidoreductases can be selected from types KRED-NADH-110 from Codexis Inc.

For the formation of the (S)-2-bromo-5,6,7,8-tetrahydro-[1,2,4]triazole[1,5-a]pyridin-8-ol of the formula (S)-Ib the following oxidoreductases have been proved to be useful.

NADPH-dependent oxidoreductases can be selected from types ADH-153 from Johnson Matthey or NADH dependent oxidoreductases can be selected from types ADH-109, ADH-110, ADH-132 and ADH-172 from c-LEcta.

For the formation of the (R)-2-bromo-5,6,7,8-tetrahydro-[1,2,4]triazole[1,5-a]pyridin-8-ol of the formula (R)-Ib the following oxidoreductases have been proved to be useful.

NADPH-dependent oxidoreductases can be selected from types KRED-425 from Codexis Inc, from types ADH-19, ADH-20 and ADH-61 from Johnson Matthey.

For the formation of the (S)-2-bromo-6,7,8,9-tetrahydro-5H-[1,2,4]triazole[1,5-a]azepin-9-ol (S)-Ic the following oxidoreductases have been proved to be useful.

NADPH-dependent oxidoreductases can be selected from types ADH-153 from Johnson Matthey, from types KRED-P2-D11, KRED-464 from Codexis Inc. or NADH dependent oxidoreductases can be selected from types ADH-109, ADH-110, ADH-132 and ADH-172 from c-LEcta.

For the formation of the (R)-2-bromo-6,7,8,9-tetrahydro-5H-[1,2,4]triazole[1,5-a]azepin-9-ol (R)-Ic the following oxidoreductases have been proved to be useful.

NADPH-dependent oxidoreductases can be selected from types KRED-425 from Codexis Inc., from types ADH-19, ADH-20 and ADH-61 from Johnson Matthey.

The chiral pyrrolo triazole alcohol of the formula

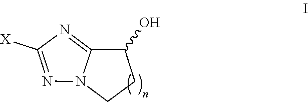

wherein
X is a halogen atom and
n is an integer of 1, 2 or 3 and the spiral bond “ [symbol] ” stands for “ [symbol] ” or for “ [symbol] ” or mixtures of the enantiomers.
are novel compounds and therefore constitute a further embodiment of the invention.

In a preferred embodiment
X is chlorine or bromine, preferably bromine and
n is 1, 2 or 3 and the spiral bond “ [symbol] ” stands for “ [symbol] ” or for “ [symbol] ” or mixtures of the enantiomers.

In a further preferred embodiment
a) X is bromine and n is 1 and the spiral bond “ [symbol] ” stands for “ [symbol] ”,
b) X is bromine and n is 1 and the spiral bond “ [symbol] ” stands for “ [symbol] ”,
c) X is bromine and n is 2, and the spiral bond “ [symbol] ” stands for “ [symbol] ”,
d) X is bromine and n is 2, and the spiral bond “ [symbol] ” stands for “ [symbol] ”,
e) X is bromine and n is 3, and the spiral bond “ [symbol] ” stands for “ [symbol] ”,
f) X is bromine and n is 3, and the spiral bond “ [symbol] ” stands for “ [symbol] ”.

### EXAMPLES

Abbreviations:

| | |
|---|---|
| % a/a: | (Area under peak of compound (a)/combined areas under all |
| | compound peaks) × 100 |
| Sel | Selectivity (% a/a product/conversion) |
| Conv | Conversion (% a/a product/(% a/a starting material) |
| MeTHF | 2-Methyl tetrahydrofuran |
| THF | Tetrahydrofuran |
| DCM | Dichloromethan |
| MeCN | Acetonitrile |
| EtOAc | Ethylacetate |
| PC | Propylen carbonate |
| Temp | Temperature |
| DMF | N,N-dimethylformamide |
| CDCl₃ | Deuterated chloroform |
| DMSO-d₆ | Deuterated dimethylsulfoxide |
| DMSO | dimethylsulfoxide |
| LC/MS | Liquid chromatography - mass spectrometry |
| HPLC | High performance liquid chromatography |
| NaOH | Sodium hydroxide |
| TBAB | Tetrabutyl ammonium bromide |
| NMR | Nuclear magnetic resonance |
| CSTR | Continuous stirred tank reactor |
| MS | Mass spectrometry |
| KRED | Ketoreductase/Oxidoreductase |
| NAD+ | Nicotniamide adenine dinucleotide |
| NADP⁺ | Nicotinamide adenine dinucleotide phosphate |
| eq | Equivalent |
| Tres | Resident time |
| HTE | High throughput experimentation |
| rt | Room temperature |
| Rct | Reaction time |
| IT | Inner temperature |
| TJ | Jacket temperature |
| rt | Room temperature |
| h | Hour(s) |
| min | Minute(s) |
| V | Volume |
| rcf | Relative centrifugal force |

Ru-Catalysts

| Num- | | |
|---|---|---|
| ber | Abbreviation | Structure |
| Ru- 292 | [Ru(Cl)2((S)-3,5-tBu- MeOBIPHEP)(H2NCH2py)] | 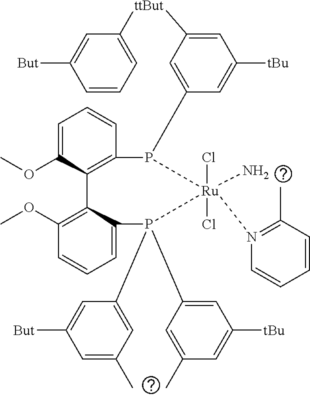 |
| Ru- 401 | [Ru((S)-DAIPENA)(Cl)((S)-3,5-Xyl- BINAP)] | 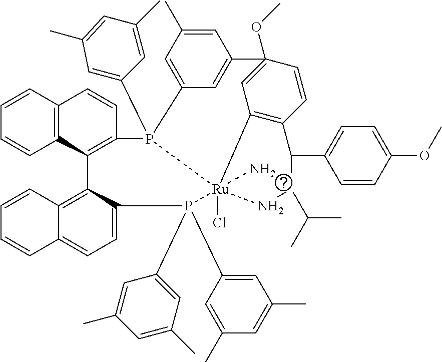 |
| Ru- 425 | [Ru(Cl)((R)-BINAP)((R,R)-2-PPh2-1,2- Ph2-ethylamine)](BF4) | 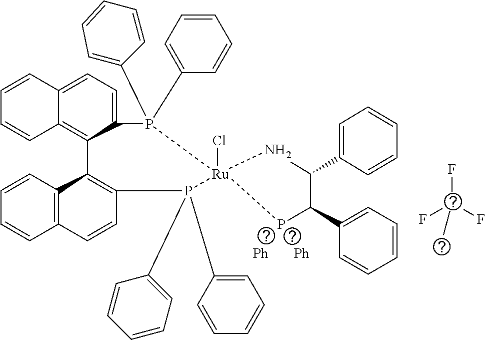 |
| Ru- 461 | [Ru((S,S)-Ts-DPEN)(Cl)(p-cymene)] | 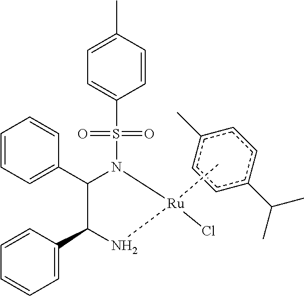 |
| Ru- 466 | [Ru((R,R)-Ts-DPEN)(Cl)(p-cymene)] | 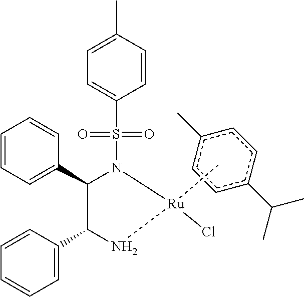 |
| Ru- 478 | [Ru((R,R)-Ts-DACH)(Cl)(p-cymene)] | 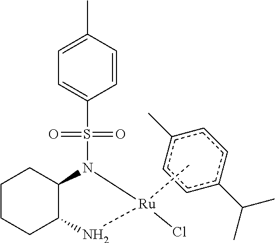 |
| Ru- 498 | [Ru(S,S)-Ts-DPEN)(p-cymene)]OTf | 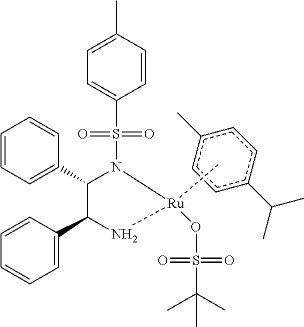 |
| Ru- 505 | [Ru((S,S,S)-Cs-DPEN)(Cl)(p-cymene)] | 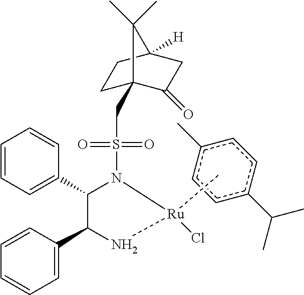 |
| Ru- 517 | [Ru((S,S)-Teth-Ts-DPEN)(Cl)] | 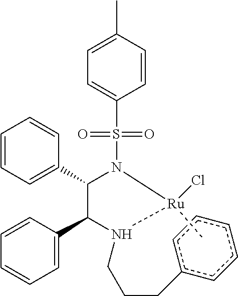 |
| Ru- 525 | [Ru((R,R)-Ts-DPEN)(Cl)(1,4-Cy₂- benzene)] | 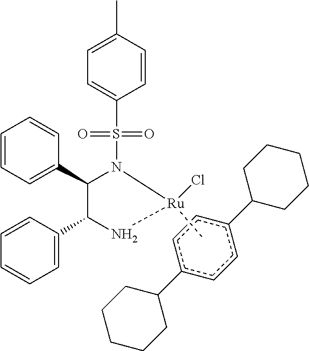 |
| Ru- 701 | [Ru((R,R)-Ms-DENEB)(Cl)] | 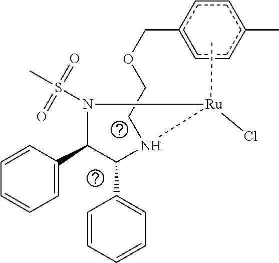 |
| Ir-15 | [Ir(Cp*)((R,R)-Ts-DPEN)(Cl) | 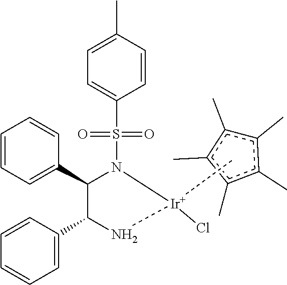 |
| Ir-29 | [Ir(cp*)((S,S)-Ms-DPEN-H)(OTf)] CAS No 917756-11-7 | 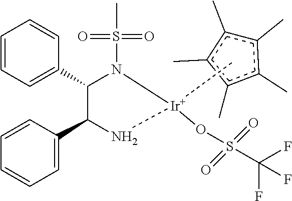 |
| Ir- 166 | [Ir(Cl)(H)2((Ss,S,S)-DTB-chf- SpiroPAP-3-Me)] | 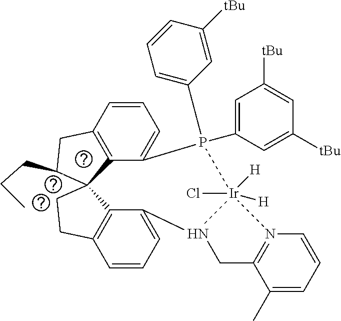 |

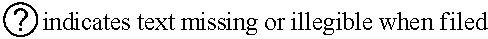

All solvents, reagents and compounds were purchased and used without further purification unless stated otherwise. All catalysts and ligands were commercially available e.g. from Jiuzhou Pharma, Sinocompound, Johnson Matthey or catalogue suppliers such as e.g. Strem or Sigma-Aldrich.

### Analytical Methods:

### NMR Methods:

¹H-NMR spectra were measured on Bruker AV 600 MHz, 400 MHz or 300 MHz spectrometer equipped with a DCH cryo probe in CDCl₃ or DMSO-d6 solutions at 25° C. with chemical shifts (δ) reported in ppm employing trimethylsilyl chloride as internal standard (δ=0 ppm).

### LC/MS Method:

LC/MS method to determine the conversions from II to IVa-c and IVa-c to Va-c and the purities of IVa-c and Va-c:
System: UPLC, Waters, Photodiode Array detector (PDA, Waters). Evaporative light scattering detector (ELSD, VWR 90 LT). LC: Stationary phase: Agilent Zorbax EclipsePlus C18 RRHT, L=30 mm, ID=2.1 mm, 1.8 μm, Eluent: A) Water 0.1% formic acid; B) MeCN 0.07% formic acid. Pump program: 97 A: 3 B, gradient to 13 A: 87 B over 2 min incl. UV Spectra. Run time: 2 min. Flow: 1 mL/min. Column oven temperature: 50° C. Injection volume: 2 μl. MS: Single quadrupole (Waters SQDO1). m/z 150-900. Detection: PDA 210-400 nm. Retention times: II: 0.65 min IVa: 0.93 min, IVb: 1.08 min, IVc: 1.15 min, Va: 0.70 min, Vb: 0.61 min, Vc: 0.69 min.

### HPLC Methods:

a) HPLC method to determine the conversion from Va to Ia and the purity and enantiomeric excess of Ia:
System: Agilent 1290. Stationary phase: Chiralpak IBN-3, L=150 mm, ID=4.6 mm, 3 μm, Eluent: A) n-heptane, B) EtOH. Pump program: 75 A: 25 B for 10 min. Run time: 10 min. Flow: 1 mL/min. Column oven temperature: 25° C. Injection volume: 10 μl. Detection: DAD 205 nm. Retention times: Va: 13.80 min, (S)-Ia: 3.72 min, (R)-Ia: 4.36 min.
b) HPLC method to determine the conversion from Vb to Ib and the purity and enantiomeric excess of Ib:
System: Agilent 1290. Stationary phase: Chiralpak IBN-3, L=150 mm, ID=4.6 mm, 3 μm. Eluent: A) n-heptane, B) EtOH. Pump program: 95 A: 5 B for 10 min, gradient to 45 A: 55 B over 5 min then hold for 2 min, gradient to 95 A: 5 B over 0.1 min then hold for 2.9 min. Run time: 20 min. Flow: 1 mL/min. Column oven temperature: 20° C. Injection volume: 6 μL. Detection: DAD 210 nm. Retention times: Vb: 17.0 min, (S)-Ib: 9.0 min, (R)-Ib: 9.7 min.
c) HPLC method to determine the conversion from Vc to Ic and the purity and enantiomeric excess of Ic:
System: Agilent 1290. Stationary phase: Chiralpak AD-3, L=150 mm, ID=4.6 mm, 3.0 m. Eluent: A) n-heptane, B) EtOH. Pump program: 90 A: 10 B for 10 min. Run time: 10 min. Flow: 1 mL/min. Column oven temperature: 25° C. Injection volume: 10 μL. Detection: DAD 205 nm. Retention times: Vc (keto/enol form): 5.70 min and 6.60 min, (S)-Ic: 3.47 min, (R)-Ic: 3.81 min.

### Optical Rotation Method:

The optical rotation values were obtained using a Anton Paar MCP-500 instrument. The optical rotations were measured in methanol at 20° C. at the indicated concentration.

### Reaction Schemes

### Synthesis of Chiral Alcohols Ia-c

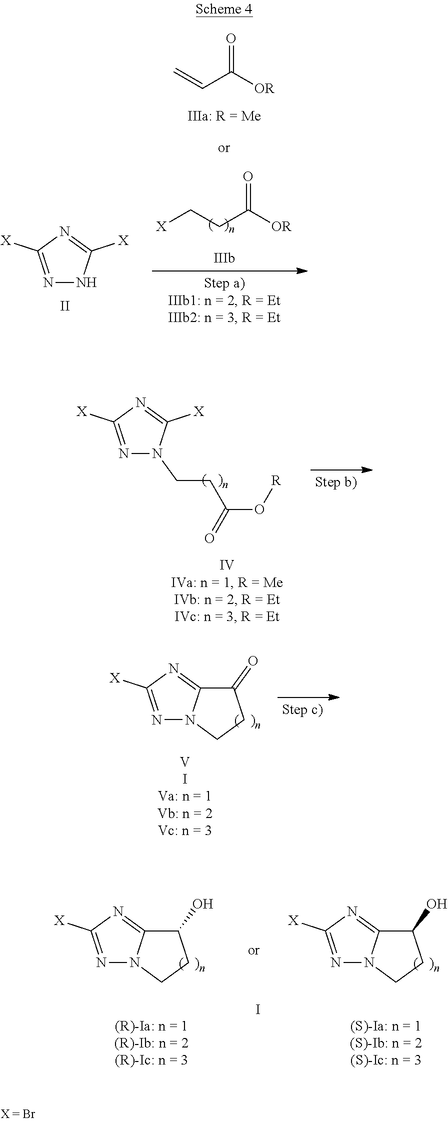

### Ring Expansion Reactions of Va and Vb

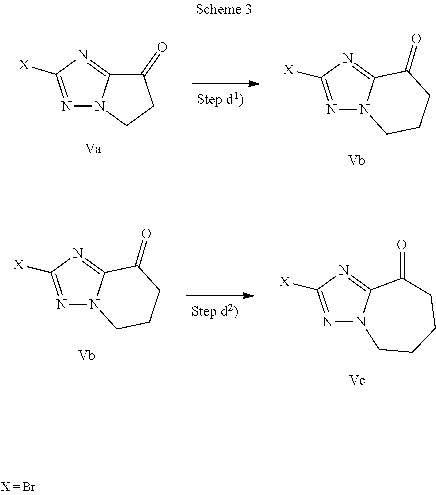

### Step A: Synthesis of Esters IVa-c

### Example 1

### a) Synthesis of 3-(3,5-dibromo-1,2,4-triazole-1-yl)propionic acid methyl ester (IVa)

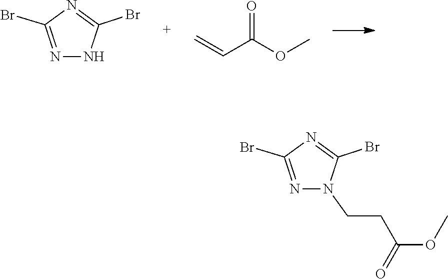

3,5-Dibromo-1H-1,2,4-triazole (40.00 g, 176.32 mmol, 1.00 eq) was suspended in MeTHF (90 mL). To the suspension it was added triethylamine (5.35 g, 52.90 mmol, 0.30 eq). After stirring for 5 min at rt, methyl acrylate (15.94 g, 16.78 mL, 185.14 mmol, 1.05 eq) was added over 4 h at 70° C. and the solution was stirred further at 70° C. for 20 h. The solution was cooled to 20° C., washed with aqueous 2 M HCl (27.24 g, 0.30 eq) followed by water (20 mL) wash. The organic phase was evaporated under vacuum to give the title compound (55.60 g, 92.0% purity, 92.7% yield) as a colorless to yellowish oil.

LC/MS: 313.9, 315.9 [M+H]⁺, ESI pos.

¹H-NMR (300 MHz, DMSO-d6): δ 4.37 (t, J=6.49 Hz, 2H), 3.60 (s, 3H), 2.94 (t, J=6.49 Hz, 2H).

### b) Synthesis of 3-(3,5-dibromo-1,2,4-triazole-1-yl)propionic acid methyl ester (IVa) on a Manufacturing Scale

3,5-Dibromo-1H-1,2,4-triazole (131.0 kg, 577 mol, 1.00 eq) was suspended in 2-MeTHF (160 kg). To the light suspension triethylamine (17.1 kg, 170 mmol, 0.30 equiv) was added and the mixture was warmed to IT 70° C. Methyl acrylate in 2-MeTHF (51.9 kg, 602 mol, 1.05 equiv in 110 kg 2-MeTHF) was added over 4-hour period. Reaction mixture was stirred further 24 h at IT 70° C. Conversion was confirmed by GC analysis (typically >98.5a %). Reaction mixture was washed with HCl (25.2 kg 25% HCl+60 kg water) at 20° C. and with water (65 kg) at 35° C. Organic phase was distilled azeotropically (200 to 100 mbar) to remove water until <200 ppm is reached (ca. 650 kg 2-MeTHF). The dried organic phase was diluted with 2-MeTHF and toluene (overall 1528 kg) to give the title compound as ca. 8 wt % solution.

### Example 2

### Synthesis of 4-(3,5-dibromo-1,2,4-triazole-1-yl)butyric acid ethyl ester (IVb)

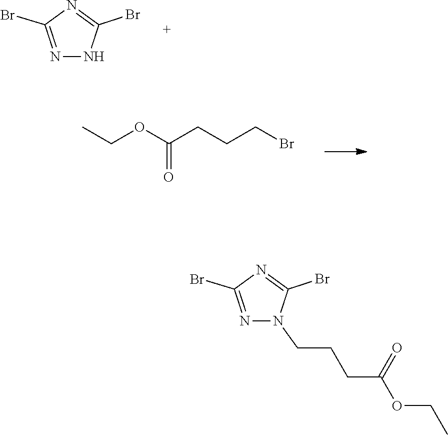

3,5-Dibromo-1H-1,2,4-triazole (24.70 g, 108.88 mmol, 1.00 eq) was dissolved in MeCN (250 mL) and DMF (25 mL). To the solution it was added potassium carbonate (37.62 g, 272.19 mmol, 2.50 eq). After stirring for 5 min at rt, ethyl 4-bromobutyrate (23.36 g, 17.30 mL, 119.77 mmol, 1.10 eq) was added and the suspension was stirred at 75° C. for 3.5 h when LC/MS indicated that the reaction reached full conversion. The suspension was allowed to get cold, filtered and the filter cake was washed several times with EtOAc (250 mL) to ensure that the product was collected in the solution. The solution was evaporated under vacuum to remove all solvents (including DMF). The crude product was purified by silica gel chromatography (eluent: EtOAc/n-heptane from 5-40%) to obtain the title compound (32.80 g, 93.0% purity, 86.6% yield) as a colorless oil.

LC/MS: 341.9, 343.9 [M+H]⁺, ESI pos.

¹H-NMR (300 MHz, CDCl₃): δ 4.24 (t, J=6.9 Hz, 2H), 4.15 (q, J=7.3 Hz, 2H), 2.41-2.34 (m, 1H), 2.25-2.14 (m, 2H), 1.27 (t, J=7.2 Hz, 3H).

### Example 3

### Synthesis of 5-(3,5-dibromo-1,2,4-triazole-1-yl)valeric acid ethyl ester (IVc)

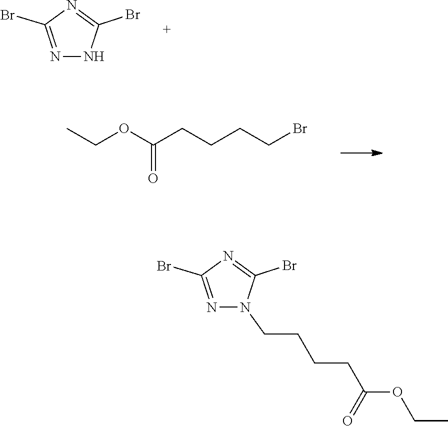

3,5-Dibromo-1H-1,2,4-triazole (20.00 g, 88.16 mmol, 1.00 eq) was dissolved in THF (70.53 mL). To the solution it was added potassium carbonate (12.43 g, 89.92 mmol, 1.02 eq) and after stirring for 5 min at rt, ethyl 5-bromovalerate (18.80 g, 13.79 mL, 89.92 mmol, 1.02 eq) was added and the suspension was stirred at 60° C. for 20 h. The suspension was allowed to cool to rt and the solids were filtered off over a funnel with a fritted disk (G3) and the solid was washed with THF (100 mL). The solution was evaporated under vacuum (200 mbar to 10 mbar) at 40° C. to yield the title compound (33.00 g, 90% purity, 94.9% yield) as colourless oil.

LC-MS 355.9/357.9 [M+H]⁺, ESI pos.

¹H NMR (400 MHz, DMSO-d₆) δ ppm 1.11-1.24 (m, 3H) 1.45-1.59 (m, 2H) 1.73-1.87 (m, 2H) 2.27-2.40 (m, 2H), 3.98-4.11 (m, 2H) 4.11-4.22 (m, 2H) ppm.

### Step B: Synthesis of Ketones Va-c

### Example 4

### a) Synthesis of 2-bromo-5,6-dihydropyrrolo[1,2-b][1,2,4]triazole-7-one (Va)

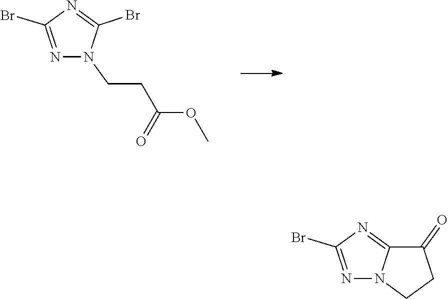

In a continuous-stirred tank reactor (CSTR) set-up 3-(3,5-dibromo-1,2,4-triazole-1-yl)propionic acid methyl ester (20.00 g, 63.91 mmol, 1.00 eq) was dissolved in MeTHF (357 mL) to give a feed A solution (0.173 M). Feed A (4.59 mL/min) was dosed in parallel together with n-hexyllitihum (2.5 M in n-heptane, 22.93 g, 83.08 mmol, 1.30 eq, 0.41 mL/min) into CSTR1 (IT−60° C., Tres 10 min). Reaction mixture was transferred from CSTR1 to CSTR2 (IT+10° C., Tres 10 min) where citric acid solution (30 wt % in water, 127.82 mmol, 2.00 eq, 0.987 mL/min) was added. Biphasic reaction mixture was transferred continuously (within 80 min) from CSTR2 into batch reactor 3. After the reaction ca. 480 mL of biphasic reaction mixture was collected in the batch reactor 3. Aqueous phase was separated and the organic phase was washed with water (60 mL). The organic phase was concentrated in vacuum to ca. 50 mL volume. n-Heptane (15 mL) was added and suspension was cooled to 0° C. The solids were filtered off over a funnel with a fritted disk (G3) and the solid was washed with an ice cold mixture of MeTHF/n-heptane (1:2 v/v, 25 mL) dried under vacuum to yield the title compound (5.86 g, 91.0% purity, 45.4% yield) as a dark brown solid.

LC/MS: 201.9, 203.9 [M+H]⁺, ESI pos.

¹H-NMR (300 MHz, DMSO-d6): δ 4.48 (t, J=5.19 Hz, 2H), 3.24 (t, J=5.19 Hz, 2H).

### b) Synthesis of 2-bromo-5,6-dihydropyrrolo[1,2-b][1,2,4]triazole-7-one (Va) on a Manufacturing Scale

3-(3,5-dibromo-1,2,4-triazole-1-yl)propionic acid methyl ester (ca. 8.00 wt % in 2-MeTHF/toluene)—Feed A (212 g/min) was dosed in parallel together with n-hexyllitihum (2.5 M in n-heptane, 18 g/min, 1.20 equiv) into CSTR1 (IT −60° C., Tres 10 min). Reaction mixture was transferred from CSTR1 to CSTR2 (IT 0 to +10° C., Tres 10 min) where citric acid solution (30 wt % in water, 50 g/min, 1.5 equiv) was added. Biphasic reaction mixture was transferred continuously from CSTR2 into batch reactor 3. The process was run until all the feed A was consumed. The aqueous phase was separated and the organic phase was washed with water (130 kg). The organic phase was concentrated in vacuum (maximum JT 35° C.) to ca. 300 L volume (target 20-25 wt % of 2-MeTHF). The formed suspension was cooled to −10° C. and stirred further at −10° C. for minimum 2 hours. The solids were filtered off over a funnel with a fritted disk (G3) and the solid was washed with an ice-cold toluene (260 kg) dried under vacuum to yield the title compound (65.4 kg, 90% Assay, isolated yield 50.5%) as a brown solid.

### Example 5

### Synthesis of 2-bromo-6,7-dihydro-5H-[1,2,4]triazole[1,5-a]pyridin-8-one (Vb)

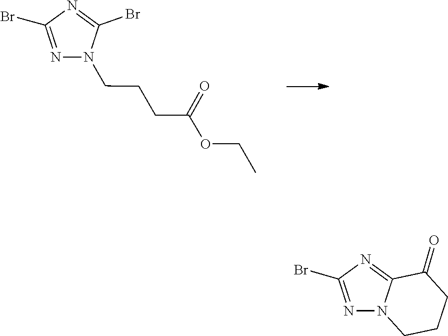

In a CSTR set-up, 4-(3,5-dibromo-1,2,4-triazole-1-yl)butyric acid ethyl ester (31.00 g, 90.91 mmol, 1.00 eq) was dissolved in MeTHF (482 mL) to give a feed A solution (0.182 M). Feed A (6.00 mL/min) was dosed in parallel together with n-hexyllithium (2.5 M in n-heptane, 30.11 g, 109.09 mmol, 1.20 eq, 0.54 mL/min) into CSTR1 (IT −60° C., Tres 10 min). The reaction mixture was transferred from CSTR1 to CSTR2 (IT+10° C., Tres 10 min) where acetic acid solution (10 wt % in water, 118.18 mmol, 1.30 eq, 0.88 mL/min) was added. The biphasic reaction mixture was transferred continuously (within 80 min) from CSTR2 into batch reactor 3 (overall 594 mL of the biphasic reaction mixture). The aqueous phase was separated and organic phase was washed with water (60 mL). The organic phase was concentrated in vacuum to ca. 50 mL volume. n-Heptane (15 mL) was added and suspension was cooled to 0° C. The solids were filtered off over a funnel with a fritted disk (G3) and the solid was washed with an ice cold mixture of MeTHF/n-heptane (1:5 v/v, 25 mL) dried under vacuum to yield the title compound (4.20 g, 98.0% purity, 21.4% yield) as pale yellow solid.

LC/MS: 215.9, 217.9 [M+H]⁺, ESI pos.

¹H-NMR (300 MHz, DMSO-d6): δ 4.40 (t, J=6.1, 2H), 2.77 (t, J=6.5 Hz, 2H), 2.35 (pent., J=6.5, 6.1 Hz, 2H).

### Example 6

### Synthesis of 2-bromo-5,6,7,8-tetrahydro-[1,2,4]triazole[1,5-a]azepin-9-one (Vc)

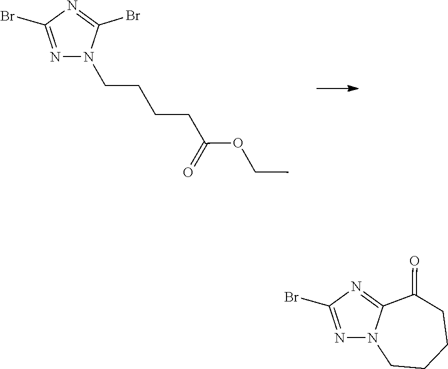 [SMILES: CCOC(=O)CCCCn1nc(Br)nc1Br ; O=C1CCCCn2nc(Br)nc21]

In a CSTR set-up as outlined in, 5-(3,5-dibromo-1,2,4-triazole-1-yl)valeric acid ethyl ester (30.80 g, 86.75 mmol, 1.00 eq) was dissolved in MeTHF (570.2 mL) to give feed A solution (0.130 M). Feed A (4.00 mL/min) was dosed in parallel together with n-hexyllithium (2.5 M in heptanes, 41.64 mL, 104.1 mmol, 1.20 eq, 0.25 mL/min) in the CSTR 1 (IT −60° C., Tres 10 min). Reaction mixture was transferred from CSTR1 to CSTR2 (IT+5° C., Tres 10 min) where acetic acid solution (10 wt % in water, 127.82 mmol, 2.00 eq, 0.625 mL/min) was added. The biphasic reaction mixture was transferred continuously (within 150 min) from CSTR2 into batch reactor 3 (overall 731 mL of biphasic reaction mixture was collected). The aqueous phase was separated and the organic phase was washed with water (60 mL). The organic phase was concentrated in vacuum to give crude 2-bromo-5,6,7,8-tetrahydro-[1,2,4]triazole[1,5-a]azepin-9-one. The product was purified by SiliaSep™ HP 25-40 μm silica column chromatography (EtOAc/n-heptane) followed by the re-purification by C18-silica gel chromatography (water/MeCN) to afford the title compound (315 mg, 95% purity, 3.1% yield) as a white solid.

LC/MS: 229.98, 231.97 [M+H]⁺, ESI pos.

¹H NMR (600 MHz, CDCl₃) δ ppm 4.48-4.62 (m, 2H), 2.89-2.99 (m, 2H), 2.16-2.28 (m, 2H), 2.02-2.12 (m, 2H) ppm.

### Step D: Synthesis of Ketones Vb-c

### Example 7

### Synthesis of 2-bromo-6,7-dihydro-5H-[1,2,4]triazole[1,5-a]pyridin-8-one (Vb)

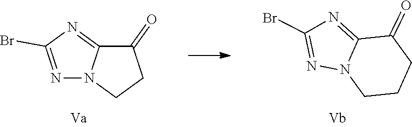 [SMILES: O=C1CCn2nc(Br)nc21 ; O=C1CCCn2nc(Br)nc21]

To a solution of 2-bromo-5,6-dihydropyrrolo[1,2-b][1,2,4]triazole-7-one (0.50 g, 2.48 mmol, 1.00 eq) dissolved in DCM (10 mL) was added dropwise 2 M trimethylaluminum 2M in toluene (1.49 mL, 2.97 mmol, 1.20 eq) at −78° C. followed by trimethylsilyl diazomethane in diethylether (1.36 mL, 2.72 mmol, 1.10 eq) that was added to the reaction mixture at −78° C. The reaction was stirred for 3 h at −20° C. (when LC/MS showed the formation of product) and a 1 M HCl solution was added. The solution was extracted two times with DCM. The organic layers were dried over MgSO₄ and concentrated to dryness. The crude material was purified by flash chromatography on silica gel to obtain the title compound (55 mg, 98.0% purity, 9.6% yield) as light brown solid.

LC/MS: 215.96, 217.98 [M+H]⁺, ESI pos.

¹H-NMR (300 MHz, CDCl₃): δ 4.47 (m, 2H), 2.86 (m, 2H), 2.48 (m, 2H).

### Example 8

### Synthesis of 2-bromo-5,6,7,8-tetrahydro-[1,2,4]triazole[1,5-a]azepin-9-one (Vc)

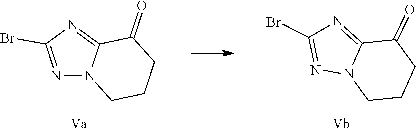 [SMILES: O=C1CCCn2nc(Br)nc21 ; O=C1CCCn2nc(Br)nc21]

2,6-Di-tert-butyl-4-methylphenol (2.24 g, 10.18 mmol, 2.20 eq) was dissolved in DCM (10 mL) and 2 M trimethylaluminum 2M in toluene (2.78 mL, 5.55 mmol, 1.20 eq) was added at rt. The mixture was stirred for 1 h and after cooling to −78° C., 2-bromo-6,7-dihydro-5H-[1,2,4]triazole[1,5-a]pyridin-8-one (1.00 g, 4.63 mmol, 1.00 eq) was added solved in dichloromethane (10 mL) followed by a 2 M solution of TMS-diazomethane 2M in diethylether (2.55 mL, 5.09 mmol, 1.10 eq). The reaction was stirred at −78° C. for 2 h until LC/MS showed the formation of the product, therefore a 1M HCl solution was added. The reaction was extracted two times with DCM and the organic layers were dried over MgSO₄ and concentrated under vacuum to dryness. The crude material was purified by flash chromatography on silica gel (40 g, EtOAc in n-heptane 0-20%). The obtained compound was further purified again with flash chromatography on C18 (50 g, water in MeCN 10-95%) to obtain the title compound (106 mg, 95.0% purity, 10.0% yield) as light brown solid.

LC/MS: 229.98, 231.97 [M+H]⁺, ESI pos.

¹H-NMR (300 MHz, CDCl₃): δ 4.54 (m, 2H), 2.93 (m, 2H), 2.23 (m, 2H), 2.09 (m, 2H).

### Step C: Synthesis of Alcohols Ia-c Via Metal Catalyzed Reduction

### Example 9.1

### Synthesis of (S)-2-bromo-6,7-dihydro-5H-pyrrolo[1,2-b][1,2,4]triazole-7-ol ((S)-Ia)

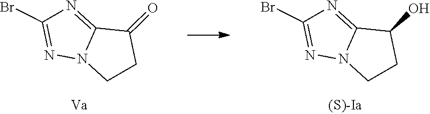 [SMILES: O=C1CCn2nc(Br)nc21 ; O[C@H]1CCn2nc(Br)nc21]

In a glovebox (<1 ppm O₂) 2-bromo-5,6-dihydropyrrolo[1,2-b][1,2,4]triazole-7-one (500 mg, 2.48 mmol, 1.00 eq) and Ru-461 (78.73 mg, 123.76 μmol, 0.05 eq) were weighed into a 50 mL Schlenk flask and dissolved in MeCN (12 mL). Then a premixed mixture of formic acid (224.06 uL, 5.84 mmol, 2.36 eq) and triethylamine (651.20 mg, 6.44 mmol, 2.60 eq) was added to the solution and rinsed with MeCN (2 mL). The flask was sealed with a septum and removed from the glovebox. The flask was connected to the argon-line and the yellow reaction mixture was stirred at Tj 31° C. in an oil bath for 20 h to give full conversion (LC/MS analysis). The yellow reaction mixture was cooled to rt and the solvent was removed to dryness under vacuum to yield the crude product. The crude product was purified by column chromatography with EtOAc/n-heptane to yield 385 mg of the title compound as a white solid. The crude product was suspended in EtOAc (2 mL) and the mixture was stirred for 10 min at rt, before n-heptane (4 mL) was added resulting in a suspension. The suspension was stirred for 30 min at rt, then cooled down to 0° C. with an ice bath and stirred for another 30 min at 0° C. The solids were filtered off over a funnel with a fritted disk (G3) and the solid was washed with an ice cold mixture of EtOAc/n-heptane (1:2 v/v, 5 mL) dried under vacuum to yield the title compound (374 mg, >99% purity, 74.0% yield, (S):(R)-Ia=>99.95:0.05) as an off-white solid.

MS (EI⁺): m/z 203.9770 [M+H]⁺.

¹H-NMR (600 MHz, DMSO-d6) δ 6.00 (br s, 1H), 5.06 (dd, J=7.5, 3.7 Hz, 1H), 4.24 (dddd, J=10.9, 8.5, 4.9, 0.9 Hz, 1H), 4.05 (ddd, J=10.9, 8.6, 5.0 Hz, 1H), 2.93 (dddd, J=13.4, 8.5, 7.7, 5.0 Hz, 1H), 2.33 (dddd, J=13.5, 8.6, 4.8, 3.9 Hz, 1H).

[α]_{D}²⁰: +10.08° (MeOH, c=1.107).

### Example 9.2

### Synthesis of (R)-2-bromo-6,7-dihydro-5H-pyrrolo[1,2-b][1,2,4]triazole-7-ol ((R)-Ia)

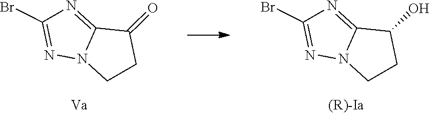 [SMILES: O=C1CCn2nc(Br)nc21 ; O[C@@H]1CCn2nc(Br)nc21]

In a glovebox (<1 ppm O₂) 2-bromo-5,6-dihydropyrrolo[1,2-b][1,2,4]triazole-7-one (100 mg, 495.03 μmol, 1.0 eq) was weighed in a 10 mL schlenk flask and suspended in MeCN (0.5 mL). Then, formic acid (57 mg, 47.5 μL, 1.24 mmol, 2.5 eq) and triethylamine (62.6 mg, 86.1 μL, 618.8 μmol, 1.25 eq) were added, followed by the addition of Ru-466 (3.15 mg, 4.95 μmol, 0.01 eq) and acetonitrile (0.5 mL). The schlenk flask was sealed with a septum, removed from the glovebox and the reaction mixture was stirred at Tj 32° C. for 19 h (LC/MS showed full conversion). The yellow reaction mixture was cooled to rt and the solvent was removed to dryness under vacuum to yield the crude product. The crude product was purified by column chromatography with EtOAc/n-heptane to yield 92 mg of the title compound as a white solid (>99% purity, 90.1% yield, (S):(R)-Ia=0.5:99.5).

[α]_{D}²⁰: −11.0° (MeOH, c=1.053).

### Examples 9.3-9.11

### Synthesis of (S)- or (R)-2-bromo-6,7-dihydro-5H-pyrrolo[1,2-b][1,2,4]triazole-7-ol (Ia)

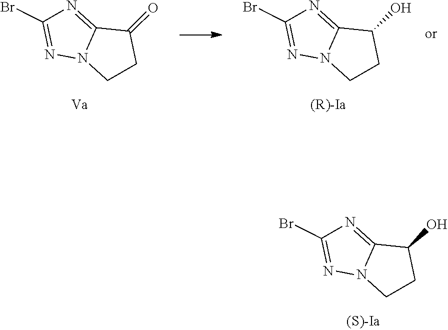 [SMILES: O=C1CCn2nc(Br)nc21 ; O[C@@H]1CCn2nc(Br)nc21 ; O[C@H]1CCn2nc(Br)nc21]

In analogy to Example 9.1 employing a HTE set-up (48-96-well plate format), Va (5 mg, 25 μmol) was reduced to Ia under the conditions as listed in Table E9.

TABLE E9 ^{a}

| | | Reducing | | Rct | Conv | Ia Sel | |
|---|---|---|---|---|---|---|---|
| Example ^{a} | Catalyst | Agent | Solv | [h] | [%] | [%] | (S)-:(R)-Ia |
| 9.3 | Ru-505 | HCO₂H/NEt₃ | Toluene | 18 | >99.9 | >99 | 99:1 |
| | | (4 eq/2 eq) | | | | | |
| 9.4 | Ir-29 | HCO₂H/NEt₃ | PC | 18 | >99.9 | 98 | 98:2 |
| | | (4 eq/4 eq) | | | | | |
| 9.5 | Ru-517 | HCO₂H/NEt₃ | EtOH | 18 | >99.9 | >99 | 96.5:3.5 |
| | | (4 eq/2 eq) | | | | | |
| 9.6 | Ru-701 | HCO₂H/NEt₃ | MeCN | 18 | >99.9 | 99 | 1.5:98.5 |
| | | (4 eq/2 eq) | | | | | |
| 9.7 | Ir-15 | HCO₂H/NEt₃ | MeTHF | 18 | >99.9 | 98 | 3:97 |
| | | (4 eq/4 eq) | | | | | |

^{a} Conditions: 5 mg scale, 0.5 mol % of catalyst, 0.5M concentration, 40° C.

| | | Reducing | Temp | | Rct | Conv | Ia Sel | |
|---|---|---|---|---|---|---|---|---|
| Example ^{b} | Catalyst | Agent | [° C.] | Solv | [h] | [%] | [%] | (S)-:(R)-Ia |
| 9.8 ^{c} | Ir-15 | H₂ | 50 | EtOH | 20 | >99.9 | 64 | 7:93 |
| | | (70 bar) | | | | | | |
| 9.9 | Ir-29 | H₂ | 40 | MeCN | 18 | >99.9 | 99 | 98.5:1.5 |
| | | (20 bar) | | | | | | |
| 9.10 | Ru-498 | H₂ | 40 | EtOH | 18 | 99 | 99 | 95.5:4.5 |
| | | (20 bar) | | | | | | |
| 9.11 ^{d} | Ru-401 | H₂ | 40 | EtOH | 6 | >99.9 | 75 | 2.5:97.5 |
| | | (20 bar) | | | | | | |

^{b} Conditions: 5 mg scale, 0.5 mol % of catalyst, solvent (10 V).
^{c} 2 mg scale, 2 mol % of catalyst, solvent (10 V).
^{d} 5 mg scale, 2 mol % of catalyst, KOtBu (0.3 eq).

### Example 10.1

### Synthesis of (S)-2-bromo-5,6,7,8-tetrahydro-[1,2,4]triazole[1,5-a]pyridin-8-ol ((S)-Ic)

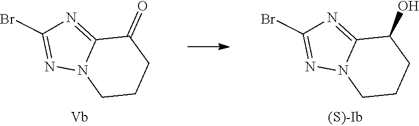 [SMILES: O=C1CCCn2nc(Br)nc21 ; O[C@H]1CCCn2nc(Br)nc21]

In a glovebox (<1 ppm O₂) a schlenk tube was loaded with 2-bromo-6,7-dihydro-5H-[1,2,4]triazolo[1,5-a]pyridin-8-one (100 mg, 462.9 μmol, 1.0 eq), toluene (2 mL), formic acid, (106.5 mg, 88.8 μL, 2.1 mmol, 5.0 eq), trimethylamine (234.2 mg, 322.6 μL, 2.3 mmol, 5.0 eq) and stirred for 2 min to give a solution. To this solution was added Ru-461 (14.72 mg, 23.14 μmol, 0.05 eq), the Schlenk tube was sealed with a septum and removed from the glovebox. The flask was connected to the argon-line and the yellow reaction mixture was stirred at Tj 42° C. in an oil bath for 4 h to give full conversion (LC/MS analysis, (S):(R)-Ib: 98.6:1.4). The yellow reaction mixture was cooled to rt and the solvent was removed to dryness under vacuum to yield the crude product (114 mg). The crude product was purified by column chromatography with EtOAc/n-heptane to yield the title compound (72 mg, 95.0% purity, 71.0% yield, (S):(R)-Ib=98.7:1.3) as a white solid.

¹H-NMR (600 MHz, DMSO-d6): δ 5.85 (br s, 1H), 4.70 (t, J=5.1, 1H), 4.14 (s, 1H), 4.00 (ddd, J=12.9, 7.9, 5.2 Hz, 1H), 2.08-2.17 (m, 1H), 1.96-2.03 (m, 1H), 1.88-1.94 (m, 1H), 1.80-1.85 (m, 1H).

[α]_{D}²⁰: +0.525° (MeOH, c=1.047).

### Example 10.2

### Synthesis of (R)-2-bromo-5,6,7,8-tetrahydro-[1,2,4]triazole[1,5-a]pyridin-8-ol ((R)-Ib

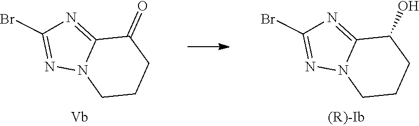 [SMILES: O=C1CCCn2nc(Br)nc21 ; O[C@@H]1CCCn2nc(Br)nc21]

In a glovebox (<1 ppm 02) an autoclave is loaded with 2-bromo-6,7-dihydro-5H-[1,2,4]triazole[1,5-a]pyridin-8-one (100 mg, 462.9 μmol, 1.0 eq), KOtBu (1.00 mg, 9.30 μmol, 0.02 eq) and Ir-15 (6.74 mg, 9.3 μmol, 0.02 eq) and ethanol (1.5 mL) to give an orange suspension. The autoclave was sealed and pressurized with 7 bar of argon and removed from the glovebox. The autoclave was connected to the hydrogenation line, the line and the autoclave was flushed with hydrogen. Then the autoclave was pressurized with 70 bar of hydrogen and stirred at Tj 52° C. for 21 h. Then the autoclave was collected to ambient temperature and the pressure was released. The autoclave was opened and sampled for analysis ((S):(R)-Ib=5:95). The reaction mixture was transferred to a round-bottomed flask and the solvent was removed under vacuum to yield the crude title compound in 108 mg as a reddish oil. The crude product was purified by column chromatography with EtOAc/n-heptane to yield the title compound (71 mg, 99.0% purity, 77.0% yield, (S):(R)-Ib=5.3:94.7) as a white solid.

MS (EI⁺): m/z 217.0 [M−H]⁺.

[α]_{D}²⁰: −3.836° (MeOH, c=1.040).

### Examples 10.3-10.10

### Synthesis of (S)- or (R)-2-bromo-5,6,7,8-tetrahydro-[1,2,4]triazole[1,5-a]pyridin-8-ol ((S)-/(R)-Ib)

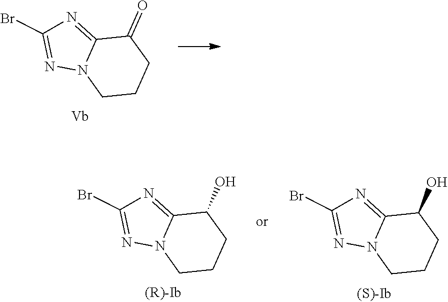 [SMILES: O=C1CCCn2nc(Br)nc21 ; O[C@@H]1CCCn2nc(Br)nc21 ; O[C@H]1CCCn2nc(Br)nc21]

In analogy to Example 10.1 employing a HTE set-up (48-96-well plate format), Vb (2 mg, 8.7 μmol) was reduced to Ib under the conditions as listed in Table E10.

TABLE E10

| | | Reducing | | Rct | Conv | Ib Sel | |
|---|---|---|---|---|---|---|---|
| | Catalyst | Agent | Solv | [h] | [%] | [%] | (S)-:(R)-Ib |
| Example ^{a} | | | | | | | |
| 10.3 | Ru-461 | HCO₂H/NEt₃ | Toluene | 4 | >99.9 | >99 | 98.5:1.5 |
| | | (5 eq/5 eq) | | | | | |
| 10.4 | Ru-478 | HCO₂H/NEt₃ | Toluene | 20 | >99.9 | >99 | 1:99 |
| | | (5 eq/5 eq) | | | | | |
| 10.5 | Ru-505 | HCO₂H/NEt₃ | Toluene | 20 | >99.9 | >99 | 99.5:0.5 |
| | | (5 eq/5 eq) | | | | | |
| 10.6 | Ru-525 | HCO₂H/NEt₃ | Toluene | 20 | >99.9 | >99 | 1.5:98.5 |
| | | (5 eq/5 eq) | | | | | |
| 10.7 | Ru-525 | NaOCHO | MeTHF | 20 | >99.9 | 96 | 4:96 |
| | | (5 eq)/TBAB | | | | | |
| | | (0.5 eq)/H₂O | | | | | |
| Example ^{b} | | | | | | | |
| 10.8 | Ir-15 | H₂ | EtOH | 16 | >99.9 | 98 | 1:99 |
| | | (70 bar) | | | | | |
| 10.9 | Ru-425 | H₂ | EtOH | 16 | >99.9 | 98 | 98.5:1.5 |
| | | (70 bar) | | | | | |
| 10.10 | Ru-517 | H₂ | EtOH | 16 | >99.9 | 96 | 98.5:1.5 |
| | | (70 bar) | | | | | |

^{a} Conditions: 2 mg scale, solvent (20 V), 40° C.
^{b} Conditions: 2 mg scale, EtOH (12.4 V), KOtBu (0.02 eq), 50° C.

### Example 11.1

### Synthesis of (S)-2-bromo-6,7,8,9-tetrahydro-5H-[1,2,4]triazole[1,5-a]azepin-9-ol ((S)-Ic)

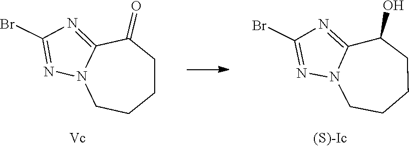 [SMILES: O=C1CCCCn2nc(Br)nc21 ; O[C@H]1CCCCn2nc(Br)nc21]

In a glovebox (<1 ppm O₂) 2-bromo-5,6,7,8-tetrahydro-[1,2,4]triazole[1,5-a]azepin-9-one (50 mg, 217.3 μmol, 1.00 eq) and Ru-461 (1.38 mg, 2.17 μmol, 0.01 eq) were weighed into a 10 mL Schlenk tube and dissolved in MeCN (1 mL). Then, a preformed mixture of MeCN, (0.5 mL), formic acid, (25 mg, 20.8 μL, 543.3 μmol, 2.5 eq) and triethylamine (27.50 mg, 37.8 μL, 271.67 μmol, 1.25 eq) was added to the solution. The flask was sealed with a septum, removed from the glovebox and the yellow reaction mixture was stirred at Tj 33° C. in an oil bath for 22 h to give full conversion (LC/MS analysis). The yellow reaction mixture was cooled to rt and the solvent was removed to dryness under vacuum to yield the crude product. The crude product was purified by column chromatography with EtOAc/n-heptane to yield the title compound (45 mg, >99% purity, 89.2% yield, (S):(R)-Ic=93.6:6.4) as a white solid.

MS (EI⁺): m/z 231 (M⁺).

¹H-NMR (600 MHz, CDCl₃): δ 4.93 (dd, J=8.8, 2.6 Hz, 1H), 4.34-4.46 (m, 1H), 4.07-4.18 (m, 1H), 2.67-3.68 (m, 1H), 2.13-2.28 (m, 1H), 1.95-2.04 (m, 1H), 1.72-1.94 (m, 4H).

[α]_{D}²⁰: +11.2960 (MeOH, c=0.108)

### Example 11.2

### Synthesis of (R)-2-bromo-6,7,8,9-tetrahydro-5H-[1,2,4]triazole[1,5-a]azepin-9-ol ((R)-Ic)

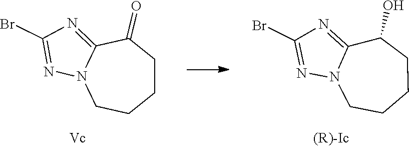 [SMILES: O=C1CCCCn2nc(Br)nc21 ; O[C@@H]1CCCCn2nc(Br)nc21]

In a glovebox (<1 ppm O₂) 2-bromo-5,6,7,8-tetrahydro-[1,2,4]triazole[1,5-a]azepin-9-one (60 mg, 260.8 μmol, 1.0 eq) and Ru-466 (1.66 mg, 2.61 μmol, 0.01 eq) were weighed into a 10 mL Schlenk tube and dissolved in MeCN (1.2 mL). Then, a preformed mixture of MeCN, (0.6 mL), formic acid, (30 mg, 25 μL, 652 μmol, 2.5 eq) and triethylamine (33 mg, 45.4 μL, 326 μmol, 1.25 eq) was added to the solution. The flask was sealed with a septum, removed from the glovebox and the yellow reaction mixture was stirred at Tj 32° C. in an oil bath for 19 h to give full conversion (LC/MS analysis). The yellow reaction mixture was cooled to rt and the solvent was removed to dryness under vacuum to yield the crude product. The crude product was purified by column chromatography with EtOAc/n-heptane to yield the title compound (56 mg, >99% purity, 92.5% yield, (S):(R)-Ic=6.2:93.8) as a white solid.

[α]_{D}²⁰: −1.669° (MeOH, c=1.067).

### Examples 11.3-11.10

### Synthesis of (S)- and (R)-2-bromo-6,7,8,9-tetrahydro-5H-[1,2,4]triazole[1,5-a]azepin-9-ol ((S)-/(R)-Ic)

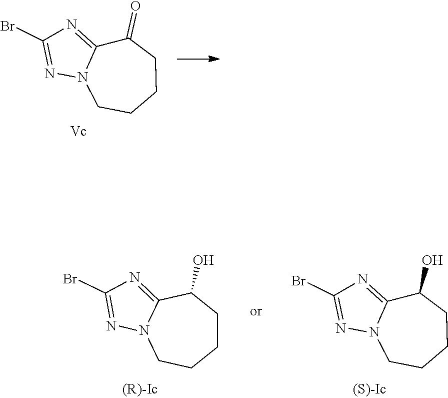 [SMILES: O=C1CCCCn2nc(Br)nc21 ; O[C@@H]1CCCCn2nc(Br)nc21 ; O[C@H]1CCCCn2nc(Br)nc21]

In analogy to Example 11.1 employing a HTE set-up (48-96-well plate format), Vc (2 mg, 8.7 μmol) was reduced to Ic under the conditions as listed in Table E11.

TABLE E11

| | | Reducing | | Rct | Conv | Ic Sel | |
|---|---|---|---|---|---|---|---|
| | Catalyst | Agent | Solv | [h] | [%] | [%] | (S)-:(R)-Ic |
| Example | | | | | | | |
| 11.2 | Ru-461 | HCO₂H/NEt₃ | PC | 16 | >99.9 | >99 | 89:11 |
| | | (4 eq/4 eq) | | | | | |
| 11.3 | Ru-478 | HCO₂H/NEt₃ | MeCN | 16 | >99.9 | >99 | 5:95 |
| | | (4 eq/4 eq) | | | | | |
| 11.4 | Ru-701 | HCO₂H/NEt₃ | MeTHF | 16 | >99.9 | 95 | 92.5:7.5 |
| | | (1 eq/1 eq) | | | | | |
| 11.5 | Ir-15 | HCO₂H/NEt₃ | MeCN | 16 | >99.9 | 92 | 9.5:90.5 |
| | | (4 eq/4 eq) | | | | | |
| 11.6 | Ir-29 | HCO₂H/NEt₃ | MeTHF | 16 | >99.9 | >99 | 93.5:6.5 |
| | | (4 eq/4 eq) | | | | | |
| Example ^{b} | | | | | | | |
| 11.7 | Ir-15 | H₂ | EtOH | 16 | >99.9 | >99 | 5.5:94.5 |
| | | (70 bar) | | | | | |
| 11.8 | Ir-166 | H₂ | EtOH | 16 | >99.9 | >99 | 1:99 |
| | | (70 bar) | | | | | |
| 11.9 | Ru-401 | H₂ | EtOH | 16 | >99.9 | 94 | 2:98 |
| | | (70 bar) | | | | | |
| 11.10 | Ru-292 | H₂ | EtOH | 16 | >99.9 | 96 | 98.5:1.5 |
| | | (70 bar) | | | | | |

^{a} Conditions: 2 mg scale, catalyst (5 mol %), solvent (10 V), 40° C., 16 h.
^{b} Conditions: 2 mg scale, EtOH (12.4 V), KOtBu (0.02 eq), 50° C.), 20 h.

### Step C: Synthesis of Alcohols Ia-c Via Enzyme Catalyzed Reductions

### Examples 12.1-12.10

### Synthesis of(S)- and (R)-2-bromo-6,7-dihydro-5H-pyrrolo[1,2-b][1,2,4]triazole-7-ol ((S)-/(R)-Ia)

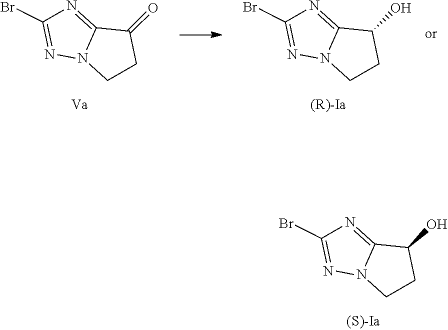 [SMILES: O=C1CCn2nc(Br)nc21 ; O[C@@H]1CCn2nc(Br)nc21 ; O[C@H]1CCn2nc(Br)nc21]

For the identification of oxidoreductases capable of the reduction of 2-bromo-5,6-dihydropyrrolo[1,2-b][1,2,4]triazole-7-one to (S)- or (R)-2-bromo-6,7-dihydro-5H-pyrrolo[1,2-b][1,2,4]triazole-7-ol a panel of oxidoreductases was screened.

The reactions were prepared in potassium phosphate buffer 100 mM at pH 7. NADP⁺ (1 g/L), NAD⁺ (1 g/L), oxidoreductases (2 g/L) as outlined in Table E12, D-glucose (100 mM), glucose dehydrogenase GDH-105 (Codexis) (0.1 g/L) were added to the reaction from stocks prepared in water. The substrate of the reaction was dissolved in dimethylsulfoxide at 100 g/L concentration and it was dispensed in the reaction at a final concentration of 5 g/L. The final volume of the reaction was 0.5 mL.

The reactions were incubated for 18 hours at room temperature. The reactions were quenched by adding 1 volume of acetonitrile. Then the reactions were centrifuged for 5 minutes at 3220 relative centrifugal force (rcf) and then the clarified solution was transferred to a fresh glass vial for analysis.

A selection of the best enzymes for the transformation is reported in Table E12:

TABLE E12

| | | Conv | Ia Sel | (S)-:(R)- |
|---|---|---|---|---|
| Example | Enzyme | [%] | [%] | Ia |
| 12.1 | ADH-109 (c-LEcta) | >99.9 | >99 | >99.9:0.1 |
| 12.2 | ADH-132 (c-LEcta) | >99.9 | >99 | >99.9:0.1 |
| 12.3 | ADH-172 (c-LEcta) | >99.9 | >99 | >99.9:0.1 |
| 12.4 | KRED-NADPH-130 (Codexis) | >99.9 | >99 | >99.9:0.1 |
| 12.5 | KRED-P1-C01 (Codexis) | >99.9 | >99 | >99.9:0.1 |
| 12.6 | KRED-P2-D11 (Codexis) | >99.9 | >99 | >99.9:0.1 |
| 12.7 | KRED-P2-D12 (Codexis) | >99.9 | >99 | >99.9:0.1 |
| 12.8 | KRED-463 (Codexis) | >99.9 | >99 | >99.9:0.1 |
| 12.9 | ADH-61 (Johnson Matthey) | >99.9 | >99 | <0.1:99.9 |
| 12.10 | KRED-NADH-110 (Codexis) | 99.3 | >99 | 0.8:99.2 |

### Example 12.11

### Synthesis of (S)-2-bromo-6,7-dihydro-5H-pyrrolo[1,2-b][1,2,4]triazole-7-ol ((S)-Ia)

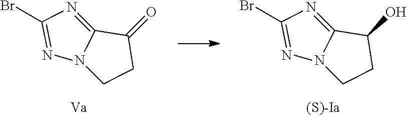 [SMILES: O=C1CCn2nc(Br)nc21 ; O[C@H]1CCn2nc(Br)nc21]

In analogy to Example 12.6, a reaction solution of 25.5 mL aqueous buffer (100 mM potassium phosphate buffer, pH 6.5) containing oxidoreductase KRED-P2-D11 from Codexis (3 mg), D-(+)-glucose monohydrate (9 mmol), commercial glucose dehydrogenase GDH-105 from Codexis (15 mg) and oxidized cofactor NADP⁺ from Roche Diagnostics (15 mg) was prepared under gentle stirring. The reaction solution was incubated at ambient temperature (23° C.) and stirred for 5 min. Afterwards reduction was started by the addition of 1.5 g (7.43 mmol) 2-bromo-5,6-dihydropyrrolo[1,2-b][1,2,4]triazole-7-one (Va) in 3 mL toluene. The pH was adjusted and kept constant during reaction by dosing NaOH 1 M via a Metrohm pH Stat (Metrohm 902 Titrando).

At ambient temperature, within 8 h complete conversion (IPC: >99.9 area % product) was achieved at constant pH, consuming 7.43 mL NaOH. Toluene was removed from the reaction by evaporation under reduced pressure. Then the reaction was filtered over filter paper. After filtration, sodium carbonate (20 g) and 2-methyltetrahydrofuran (100 mL) were added to the reaction, vigorously mixed and the phases separated spontaneously. The separated aqueous phase was extracted once again with 2-methyltetrahydrofuran (100 mL) and the combined phases were dried over MgSO₄, filtered and evaporated under vacuum at 40° C. to yield the title compound (1.15 g, >95% purity, 76.0% yield, (S):(R)-Ia>99.9:0.1%, (R)-Ia and Va not detected) as an off white solid.

LC/MS: 203. 98 (M+H)⁺. ESI pos.

¹H-NMR (600 MHz, DMSO-d6) δ 5.99 (br s, 1H), 5.05 (dd, J=6.9, 3.2 Hz, 1H), 4.23 (dddd, J=10.8, 8.5, 4.9, 0.9 Hz, 1H), 4.04 (ddd, J=10.9, 8.6, 5.0 Hz, 1H), 2.93 (dddd, J=13.4, 8.5, 7.8, 5.0 Hz, 1H), 2.32 (dddd, J=13.4, 8.6, 4.9, 3.8 Hz, 1H).

### Example 12.12

### Synthesis of (R)-2-bromo-6,7-dihydro-5H-pyrrolo[1,2-b][1,2,4]triazole-7-ol ((R)-Ia)

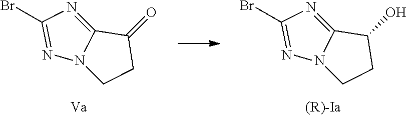 [SMILES: O=C1CCn2nc(Br)nc21 ; O[C@@H]1CCn2nc(Br)nc21]

In analogy to Example 12.12, a reaction solution of 32.1 mL aqueous buffer (100 mM potassium phosphate buffer, pH 6.5) containing oxidoreductase KRED-NADH-110 from Codexis (250 mg), D-(+)-glucose monohydrate (27.2 mmol), commercial glucose dehydrogenase GDH-105 from Codexis (250 mg) and oxidized cofactor NADP⁺ from Roche Diagnostics (250 mg) was prepared under gentle stirring. The reaction solution was incubated at ambient temperature (23° C.) and stirred for 5 min. Afterwards reduction was started by the addition of 5 g (24.75 mmol) 2-bromo-5,6-dihydropyrrolo[1,2-b][1,2,4]triazole-7-one (Va) in 7.5 mL toluene. The pH was adjusted and kept constant during reaction by dosing NaOH 1 M via a Metrohm pH Stat (Metrohm 902 Titrando). At ambient temperature within 8 h complete conversion (IPC: >99.9 area % product were achieved at constant pH, consuming 12.38 mL NaOH. Toluene was removed from the reaction by evaporation under reduced pressure. Then the reaction was filtered over filter paper. After filtration, sodium carbonate (20 g) and 2-methyltetrahydrofuran (100 mL) were added to the reaction, vigorously mixed and the phases separated spontaneously. The separated aqueous phase was extracted once again with 2-methyltetrahydrofuran (100 mL) and the combined phases were dried over MgSO₄, filtered and evaporated under vacuum at 40° C. to yield the title compound (1.64 g, >95% purity, 32.0% yield, (S):(R)-Ia=1.4:98.6, Va not detected) as an off white solid.

LC/MS: 203.98 (M+H)⁺. ESI pos.

1H NMR (600 MHz, DMSO-d6) δ ppm 5.99 (br s, 1H), 5.05 (br dd, J=6.9, 3.2 Hz, 1H), 4.23 (dddd, J=10.8, 8.5, 4.9, 0.9 Hz, 1H), 4.04 (ddd, J=10.8, 8.6, 5.0 Hz, 1H), 2.93 (dddd, J=13.4, 8.5, 7.8, 5.0 Hz, 1H), 2.32 (dddd, J=13.4, 8.6, 4.9, 3.8 Hz, 1H).

### Example 12.13

### Synthesis of (S)-2-bromo-6,7-dihydro-5H-pyrrolo[1,2-b][1,2,4]triazole-7-ol ((S)-Ia) in a Large Scale

In analogy to Example 12.11, a reaction was prepared by mixing in an inerted reactor 11.75 kg KH₂PO₄ and 11.76 K₂HPO₄, 385 g NADP⁺ disodium salt, 1.54 kg KRED-P2-D11 from Codexis in 1,100 L water. Subsequently, 240 L of isopropanol were added in the reactor. The pH of the solution was 7.0 and the solution was incubated at 22° C. The reaction was started by adding 77 kg (442 mol) of 2-bromo-5,6-dihydropyrrolo[1,2-b][1,2,4]triazole-7-one (Va) in 8 portions of 9.8 to 9.6 kg at one hour intervals. The reaction was complete after 12 hours (IPC: >99.9% conversion, 97.9% Ia, (S):(R)-Ia>99.9:0.1%).

Isopropanol and acetone were removed under reduced pressure at 55° C. until 560 kg of distillate was removed. 1150 kg of water were added and the reaction was agitated for 30 minutes. Then pH was set to 2.0 by adding 53 kg of 20% sulfuric acid in water, the mixture was then stirred for 45 minutes at 55° C. to allow enzyme precipitation.

The solution was filtered on 3M™ Zeta Plus™ filter cartridge, then the feeder was rinsed twice with a mixture of 231 kg water and 11.5 kg 20% sulfuric acid in water and this solution was also passed through the filter.

The filtered solution was heated at 100° C. and water was distilled until approximately 720 kg of solution remained in the autoclave. The temperature was lowered to 25° C., 1,150 kg of 2-methyltetrahydrofuran were added to the reactor and the compound Ia was extracted in the organic phase. The water fraction was removed and the extraction procedure was repeated twice by adding 493 kg 2-methyltetrahydrofuran. Subsequently, the solution of Ia in 2-methyltetrahydrofuran was filtered through Zetacarbon filters.

The 2-methyltetrahydrofuran solution was heated to 110° C. and the solvent was removed under vacuum until 380 kg of solution remained in the reactor. The solution was cooled to 80° C. and then further cooled to 20° C. at a rate of 20° C./hour. Then 237 kg of n-heptane were added to the reactor in 45 minutes and the solution was stirred for 2 hours. The suspension was then cooled to 0° C. at a rate of 10° C./hour and stirred for 2 hours.

The suspension was centrifuged and the crystals were washed with 264 kg of n-heptane. Finally the crystals were dried in an oven under full vacuum at 50° C.

The compound Ia was isolated with a reaction yield of 84% (IPC purity 99.7%, (S):(R)-Ia>99.9:0.1%) as an off-white crystal.

### Examples 13.1-13.9

### Synthesis of (S)- and (R)-2-bromo-5,6,7,8-tetrahydro-[1,2,4]triazole [1,5-a]pyridin-8-ol ((S)-/(R)-Ib)

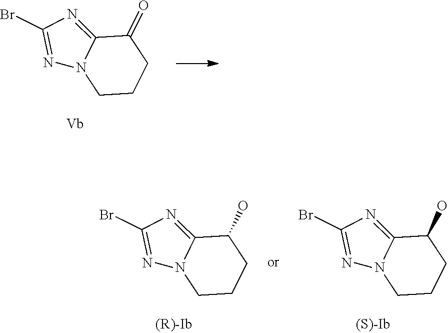 [SMILES: O=C1CCCn2nc(Br)nc21 ; O[C@@H]1CCCn2nc(Br)nc21 ; O[C@H]1CCCn2nc(Br)nc21]

For the identification of oxidoreductases capable of the reduction of 2-bromo-6,7-dihydro-5H-[1,2,4]triazole[1,5-a]pyridin-8-one to (8S) or (8R)-2-bromo-5,6,7,8-tetrahydro-[1,2,4]triazole[1,5-a]pyridin-8-ol a panel of oxidoreductases was screened. The reactions were prepared in potassium phosphate buffer 100 mM at pH 6.5. NADP⁺ (1 g/L), NAD⁺ (1 g/L), oxidoreductases (0.08 g/L), D-glucose (100 mM), glucose dehydrogenase GDH-105 (Codexis) (0.02 g/L) were added to the reaction from stocks prepared in water. The substrate of the reaction was dissolved in dimethylsulfoxide at 40 g/L concentration and it was dispensed in the reaction at a final concentration of 2 g/L. The final volume of the reaction was 0.5 mL. The reactions were incubated between 2 and 16 h at rt. The reactions were quenched by adding 1 volume of MeCN. Then the reactions were centrifuged for 5 mins at 3220 rcf and then the clarified solution was transferred to a fresh glass vial for analysis. A selection of the enzymes for the transformation is reported in Table E13:

TABLE E13

| | | Conv | Ib Sel | (S)-:(R)- |
|---|---|---|---|---|
| Example | Enzyme | [%] | [%] | Ib |
| 13.1 | ADH-172 (c-LEcta) | 99.0 | >99 | >99.9:0.1 |
| 13.2 | ADH-110 (c-LEcta) | 99.0 | >99 | 99.65:0.35 |
| 13.3 | ADH-109 (c-LEcta) | 99.0 | >99 | 99.65:0.35 |
| 13.4 | ADH-132 (c-LEcta) | 99.0 | >99 | >99.9:0.1 |
| 13.5 | ADH-153 (Johnson Matthey) | 94.0 | >99 | >99.5:0.5 |
| 13.6 | KRED-425 (Codexis) | 97.1 | >99 | <0.1:99.9 |
| 13.7 | ADH-19 (Johnson Matthey) | 97.1 | >99 | <0.1:99.9 |
| 13.8 | ADH-20 (Johnson Matthey) | 99.1 | >99 | 1.8:98.2 |
| 13.9 | ADH-61 (Johnson Matthey) | 98.0 | >99 | <0.1:99.9 |

### Examples 14.1-14.11

### Synthesis of (S)- and (R)-2-bromo-6,7,8,9-tetrahydro-5H-[1,2,4]triazole[1,5-a]azepin-9-ol ((S)-/(R)-Ic)

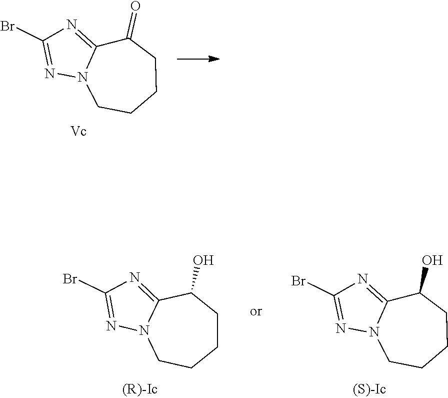 [SMILES: O=C1CCCCn2nc(Br)nc21 ; O[C@@H]1CCCCn2nc(Br)nc21 ; O[C@H]1CCCCn2nc(Br)nc21]

For the identification of oxidoreductases capable of the reduction of 2-bromo-5,6,7,8-tetrahydro-[1,2,4]triazole[1,5-a]azepin-9-one to (9S) or to (9R)-2-bromo-6,7,8,9-tetrahydro-5H-[1,2,4]triazole[1,5-a]azepin-9-ol a panel of oxidoreductases was screened. The reactions were prepared in potassium phosphate buffer 100 mM at pH 6.5. NADP⁺ (1 g/L), NAD⁺ (1 g/L), oxidoreductases (0.08 g/L), D-glucose (100 mM), glucose dehydrogenase GDH-105 (Codexis) (0.02 g/L) were added to the reaction from stocks prepared in water. The substrate of the reaction was dissolved in DMSO at 40 g/L concentration and it was dispensed in the reaction at a final concentration of 2 g/L. The final volume of the reaction was 0.5 mL. The reactions were incubated between 2 and 16 h at rt. The reactions were quenched by adding 1 volume of MeCN. Then the reactions were centrifuged for 5 min at 3220 rcf and then the clarified solution was transferred to a fresh glass vial for analysis. A selection of the enzymes for the transformation is reported in Table E14:

TABLE E14

| | | Conv | Ic Sel | (S)-:(R)- |
|---|---|---|---|---|
| Example | Enzyme | [%] | [%] | Ic |
| 14.1 | ADH-132 (c-LEcta) | >99.9 | >99 | 99.8:0.2 |
| 14.2 | ADH-117 (c-LEcta) | >99.9 | >99 | 99.6:0.4 |
| 14.3 | ADH-109 (c-LEcta) | >99.9 | >99 | 99.5:0.5 |
| 14.4 | ADH-172 (c-LEcta) | 99.9 | >99 | 99.85:0.15 |
| 14.5 | ADH-153 (Johnson Matthey) | 99.5 | >99 | 99.7:0.3 |
| 14.6 | KRED-P2-D11 (Codexis) | 99.1 | >99 | 99.5:0.5 |
| 14.7 | KRED-464 (Codexis) | 98.9 | >99 | 99.6:0.4 |
| 14.8 | KRED-425 (Codexis) | >99.9 | >99 | 1.1:98.9 |
| 14.9 | ADH-19 (Johnson Matthey) | 99.0 | >99 | <0.1:99.9 |
| 14.10 | ADH-20 (Johnson Matthey) | 98.6 | >99 | 0.5:99.5 |
| 14.11 | ADH-61 (Johnson Matthey) | 99.6 | >99 | <0.1:99.9 |

1. A process for the preparation of a chiral pyrrolo triazole alcohol of the formula I 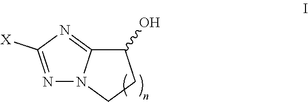
wherein X is a halogen atom, n is an integer of 1, 2 or 3 and wherein the spiral bond “ [symbol] ” stands for “ [symbol] ” or for “ [symbol] ” or mixtures of the enantiomers,
comprising the reduction of a ketone of the formula V 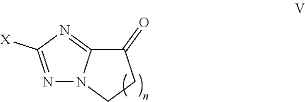
wherein X and n are as defined for formula I
a) a metal complex catalyst in the presence of a reducing agent or
b) an oxidoreductase enzyme
to form the chiral alcohol of the formula I.

2. The process of claim 1, wherein the metal complex catalyst is a ruthenium or iridium complex catalyst.

3. The process of claim 1, wherein the metal complex catalyst is selected from ruthenium or iridium catalyst complexes of any one of the following formulae: 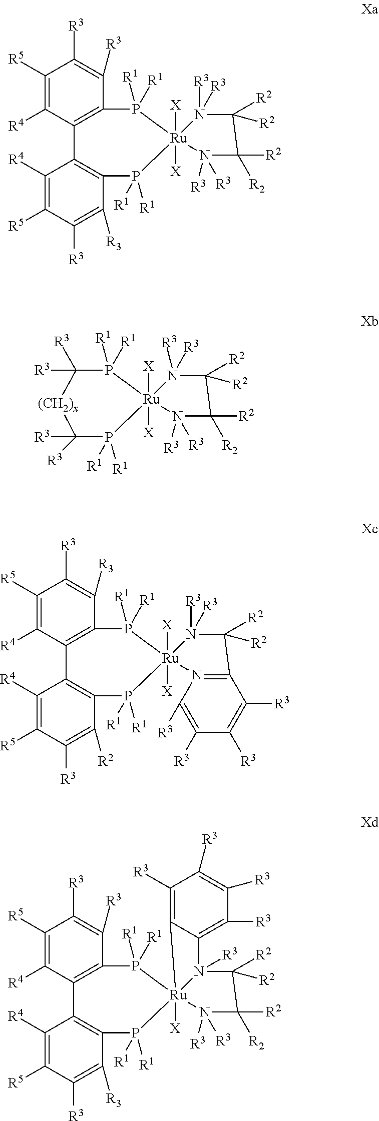 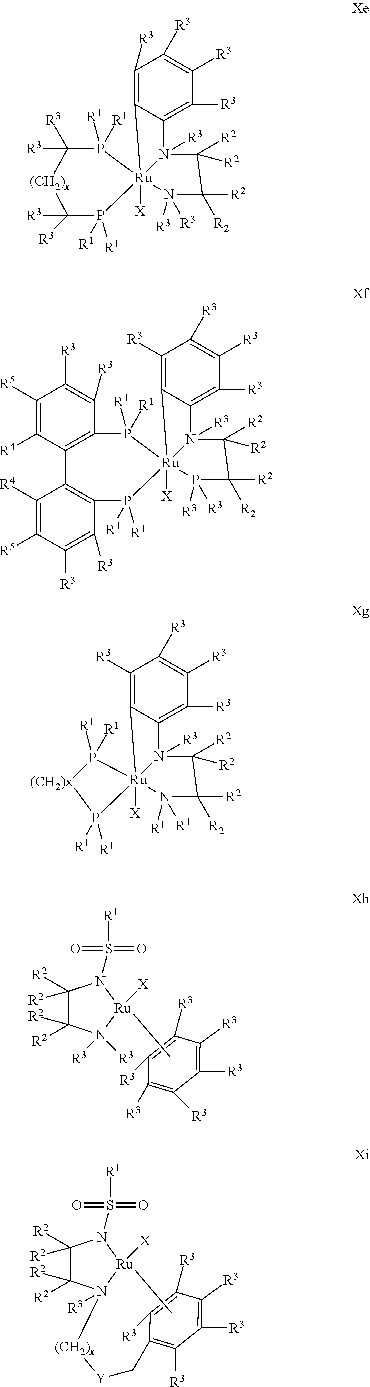 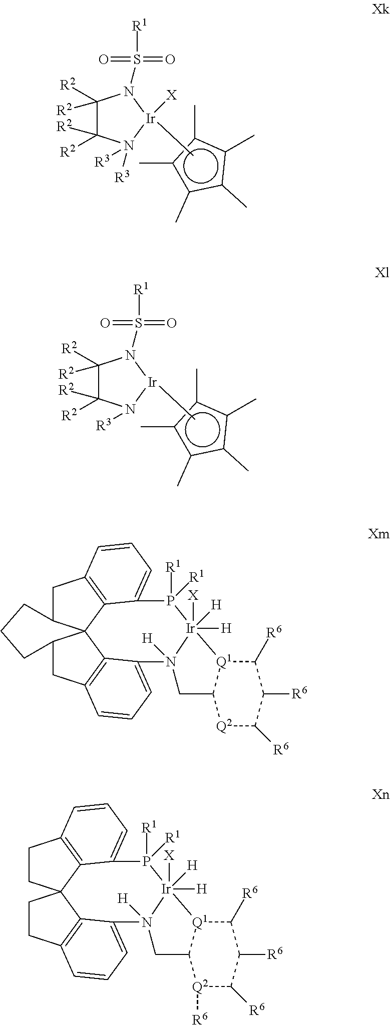
wherein, for each individual structure and independent of each other,
R¹ is, independently of each other, C₁₋₆-alkyl, C₄₋₆-cycloalkyl, phenyl or heteroaryl, optionally substituted with one or more C₁₋₆-alkyl or C₁₋₆-alkoxy;
R² is, independently of each other, hydrogen, C₁₋₆-alkyl, C₄₋₆-cycloalkyl, phenyl or heteroaryl, optionally substituted with one or more C₁₋₆-alkyl or C₁₋₆-alkoxy or two R² taken together form a ring bridged with a —(CH₂)₄— unit;
R³ is, independently of each other, hydrogen or C₁₋₆-alkyl, C₄₋₆-cycloalkyl, phenyl or heteroaryl, optionally substituted with one or more C₁₋₆-alkyl or C₁₋₆-alkoxy;
R⁴ is, independently of each other, hydrogen, C₁₋₆-alkyl, C₄₋₆-cycloalkyl, phenyl or heteroaryl, optionally substituted with one or more C₁₋₆-alkyl or C₁₋₆-alkoxy or both R⁴ are taken together to form a ring bridged with a —O—(CH₂)ₓ—O— unit;
R⁵ is, independently of each other, hydrogen, C₁₋₆-alkyl, C₄₋₆-cycloalkyl, phenyl or heteroaryl, optionally substituted with one or more C₁₋₆-alkyl or C₁₋₆-alkoxy or both neighboring R⁴ and R⁵ are taken together to form a ring bridged with a —(CH)₄— unit or a —O—(CH₂)x-O— unit;
R⁶ is, independently of each other, hydrogen or C₁₋₆-alkyl;
X is either a coordinated ligand or a counter anion selected from halogen, C₁₋₆-alkoxy, tetrahalogenoborate, tetrakis (3,5-bis(trihalogeno-C₁₋₆-alkyl) phenyl)borate, acetylacetonate, hexahalogenophosphate, p-tolylsulfonate, methansulfonate or trihalogeno methanesulfonate;
Y is oxygen or —CH₂—;
x is 1, 2 or 3;
and the dotted ring signifies an aromatic ring when Q¹ is nitrogen and Q² is carbon;
and the dotted ring signifies a cycloalkane ring wherein Q¹ and Q² are sulfur.

4. The process of t claim 3, wherein metal complex catalyst is selected from metal catalyst complexes of the formula Xh, Xk, Xm or Xn.

5. The process of claim 4, wherein the reducing agent is a mixture of formic acid and a trialkylamine or is hydrogen.

6. The process of claim 5, wherein the reaction with a mixture of formic acid and a trialkylamine takes place in the presence of an organic solvent and at a reaction temperature of 10° C. to 100° C.

7. The process of claim 5, wherein the reaction with hydrogen takes place at a hydrogen pressure of 1 bar to 100 bar and at a reaction temperature of 10° C. to 100° C. in an organic solvent.

8. The process of claim 1, wherein the selected oxidoreductase has the potential to reduce the ketone of formula V and to form the chiral pyrrolo triazole alcohol of the formula I with an enantiomeric excess of at least 90%, preferably at least 95% and more preferably at least 98%.

9. The process of claim 1, wherein the enzymatic reduction is performed in the presence of NADH or NADPH as cofactor.

10. The process of claim 9, wherein the cofactor is regenerated with a cosubstrate.

11. The process of claim 10, wherein the cosubstrate is a secondary alcohol, preferably 2-propanol.

12. The process of claim 11, wherein the enzymatic reduction is performed in an aqueous medium in the presence of an organic cosolvent at temperatures of 10° C. to 50° C.

13. The process of claim 1, wherein the ketone of the formula V can be prepared by
a) transforming a 3,5-dihalogen-1H-1,2,4-triazole of formula II 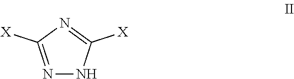
wherein X is as defined for formula I, either by
a¹) a Michael addition using an acryl ester IIIa. 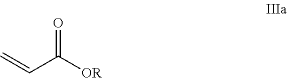
wherein R is C₁₋₄alkyl or by
a²) an alkylation with a halogen carboxylic acid alkyl ester of formula IIIb 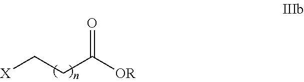
wherein R is as defined for formula IIIa, and X and n are as defined for formula I,
into a 3,5-dihalogen 1,2,4-triazole-carboxylic acid ester of formula IV 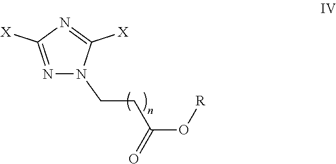
wherein R is as defined for formula IIIa or IIIb, and X and n are as defined for formula I, and
b) a ring closure of the 3,5-dihalogen 1,2,4-triazole-carboxylic acid ester of formula IV with organometallic reagents to form the ketone of the formula V

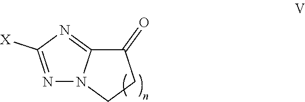

14. A chiral pyrrolo triazole alcohol of the formula I 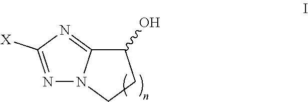
wherein X is a halogen atom and n is an integer of 1, 2 or 3 and the spiral bond “ [symbol] ” stands for “ [symbol] ” or for “ [symbol] ” or mixtures of the enantiomers.

15. The chiral pyrrolo triazole alcohol of claim 14, wherein X is chlorine or bromine, preferably bromine and n is 1, 2 or 3 and the spiral bond “ [symbol] ” stands for “ [symbol] ” or for “ [symbol] ” or for mixtures of the enantiomers.

16. The chiral pyrrolo triazole alcohol of claim 14, wherein
a) X is bromine and n is 1 and the spiral bond “ [symbol] ” stands for “ [symbol] ”,
b) X is bromine and n is 1 and the spiral bond “ [symbol] ” stands for “ [symbol] ”,
c) X is bromine and n is 2, and the spiral bond “ [symbol] ” stands for “ [symbol] ”,
d) X is bromine and n is 2, and the spiral bond “ [symbol] ” stands for “ [symbol] ”,
e) X is bromine and n is 3, and the spiral bond “ [symbol] ” stands for “ [symbol] ”, or
f) X is bromine and n is 3, and the spiral bond “ [symbol] ” stands for “ [symbol] ”.

17. A 3,5-Dihalogen 1,2,4-triazole-carboxylic acid ester of formula IV 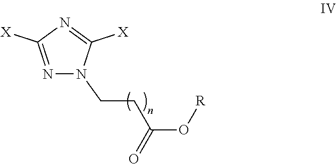
wherein R is C₁₋₄ alkyl, n is an integer of 1, 2 or 3 and X is halogen.

18. The 3,5-Dihalogen 1,2,4-triazole-carboxylic acid ester of claim 17, wherein X is bromine.

19. The 3,5-Dihalogen 1,2,4-triazole-carboxylic acid ester of claim 17, R is methyl or ethyl.
